(19) United States
(12) Patent Application Publication
(10) Pub. No.: US 2015/0025105 A1
(43) Pub. Date: Jan. 22, 2015

(54) INDOLIZINE DERIVATIVES

(71) Applicants: Praveen Rao PERAMPALLI NEKKAR, (US); Saad NAMBIL KABIR, (US)

(72) Inventors: Praveen Rao Perampalli Nekkar, Kitchener (CA); Saad Nabil Kabir, Mississauga (CA)

(21) Appl. No.: 14/377,590

(22) PCT Filed: Feb. 8, 2013
(86) PCT No.: PCT/CA2013/050104

§ 371 Date: Aug. 8, 2014

Related U.S. Application Data
(60) Provisional application No. 61/633,325, filed on Feb. 9, 2012.

(51) Int. Cl. C07D 471/04

(52) U.S. Cl. CPC C07D 471/04
USPC 514/299; 546/112; 435/184

(57) ABSTRACT

Disclosed herein are indolizine compounds of general formula I, wherein R¹-R⁷, X and Y are as disclosed in the application, useful as inhibitors of lipoprotein associated phospholipase A₂ (Lp-PLA₂) and/or 15-lipoxygenase (15-LOX), as well as methods of preparation and use thereof, and compositions thereof Advantageously, certain compounds disclosed herein are capable of inhibiting both Lp-PLA₂ and 15-LOX. Accordingly, provided herein are methods of inhibiting one or both of Lp-PLA₂ and 15-LOX, and methods of treating diseases or conditions associated with Lp-PLA₂ and/or 15-LOX.

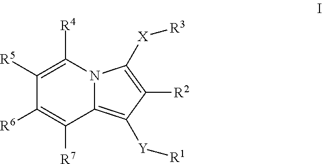

### RELATED APPLICATIONS

This application claims benefit of priority to U.S. Provisional Patent Application Ser. No. 61/633,325 filed on Feb. 9, 2012, which is hereby incorporated by reference.

### FIELD OF THE INVENTION

The present disclosure relates to a novel class of indolizine derivatives useful as inhibitors of lipoprotein associated phospholipase A₂ (Lp-PLA₂) and/or 15-lipoxygenase (15-LOX), methods of preparation and use thereof, and compositions comprising same. In some embodiments, the indolizine derivatives are dual inhibitors of Lp-PLA₂ and 15-LOX. Accordingly, the present disclosure also relates to methods of inhibiting Lp-PLA₂ and/or 15-LOX. In some embodiments, the present disclosure also relates to methods of treating a disease or condition associated with Lp-PLA₂ and/or 15-LOX, including but not limited to, cardiovascular, inflammatory and proliferative diseases and conditions.

### BACKGROUND OF THE INVENTION

Phospholipase A2 (PLA2) catalyzes the hydrolysis of membrane phospholipids resulting in the release of fatty acids, including arachidonic acid (AA), by acting on membrane phospholipids. Upon can be converted to various pro-inflammatory mediators including prostaglandins, leukotrienes and platelet-activating factor (PAF), that are known to play a major role in regulating vascular tone. There are three major subtypes of PLA2: secretory (sPLA2); cytosolic or Ca2+-activated (cPLA2); and inducible or Ca2+-independent (iPLA2). In this regard, Lp-PLA2, also known as platelet-activating factor acetylhydrolase (PAF-AH), is a Ca2+-independent PLA2 that is classified as group VIIA PLA2. Recent studies have suggested that Lp-PLA2 plays a role in the onset and progression of atherosclerosis. The enzyme Lp-PLA2 or PAF-AH (EC 3.1.1.47) was first identified from plasma, and was known to hydrolyze/inactivate PAF, a phospholipid mediator produced from macrophages, monocytes, platelets and neutrophils which is involved in inflammatory diseases including atherosclerosis. In humans, Lp-PLA2 is primarily produced in leukocytes and macrophages and is associated with circulating macrophages and low-density lipoproteins (LDL). It acts on polar phospholipids in oxidized LDL to form lysophosphatidylcholine and nonesterified phospholipids that are known to have pro-inflammatory properties by activating and recruiting macrophages/monocytes mediating plaque vulnerability, and apoptosis, leading to onset and progression of atheroma. The enzyme Lp-PLA2 is known to be involved in number of conditions such as atherosclerosis, stroke, myocardial infarction, acute coronary syndrome, coronary heart disease, peripheral arterial disease, rheumatoid arthritis, psoriasis and acute/chronic inflammation. Studies suggest that Lp-PLA2 is a target to develop novel therapeutic agents for the treatment of various diseases and conditions, including cardiovascular and inflammatory diseases and cancers. Darapladib is the first selective Lp-PLA2 inhibitor currently being tested in Phase-III clinical trials as an antiatherosclerotic agent by GlaxoSmithKline. Information on this trial is available online at http://clinicaltrials.gov/show/NCT00799903. Both Lp-PLA2 and 15-LOX are present in carotid plaque macrophages indicating their co-localization.

Lipoxygenases (LOXs) belong to a class of non-heme iron-containing enzymes that catalyze dioxygen incorporation into polyunsaturated fatty acids, such as linoleic and arachidonic acid, to form hydroperoxide products. The fatty acid metabolites of 15-lipoxygenase (15-LOX) are implicated in the oxidative modification of low-density lipoprotein (LDL) and 15-LOX mediated formation of cholesterol ester hydroperoxides, promoting plaque formation leading to atherosclerosis. The end products of arachidonic acid metabolism by 15-LOX have long been implicated in asthma, atherosclerosis, rheumatoid arthritis and in pancreatic, prostate, colorectal cancers. This supports the development of 15-LOX inhibitors as therapeutic agents. Several patents on the therapeutic application of novel 15-LOX inhibitors are reported.

It is therefore desirable to identify inhibitors of Lp-PLA₂ and/or 15-LOX. Such inhibitors would be useful for inhibiting Lp-PLA₂ and/or 15-LOX in vitro. Such inhibitors may also be useful in the treatment of diseases and conditions associated with Lp-PLA₂ and 15-LOX, such as cardiovascular and inflammatory diseases or conditions and cancers. Dual inhibitors of Lp-PLA₂ and 15-LOX may be particularly advantagoous.

### BRIEF DESCRIPTION OF PRIOR ART

WO2013013503 and US2012142717 describe Lp-PLA₂ inhibitors.

US2007049628, WO2007051982, WO2008129280 and WO200813567 describe 15-LOX inhibitors.

U.S. Pat. No. 4,751,235 describes indolizinylheptanoic acid derivatives as inhibitors of the enzyme 3-hydroxy-3-methylglutaryl coenzyme A (HMG-CoA) reductase and their application as hypocholestremic agents.

U.S. Pat. No. 6,645,976 describes indolizine acetamides, acetic acid hydrazides and gloxylamides small molecules that target the inflammatory enzyme sPLA₂ and their use in treating conditions such as septic shock, adult respiratory distress syndrome, pancreatitis, trauma, bronchial asthma, allergic rhinitis, rheumatoid arthritis, gout, glomerulonephritis and related diseases.

WO 03/042218 describes pyridinone and pyrimidinone derivatives as Lp-PLA₂ inhibitors and their use in treating atherosclerosis, diabetes, rheumatoid arthritis, stroke, myocardial infarction, reperfusion injury, acute/chronic inflammation and psoriasis.

U.S. Patent Publication No. 2004/0063753 describes pyridinone derivatives as Lp-PLA₂ inhibitors and their use in treatment of atherosclerosis, diabetes, rheumatoid arthritis, stroke, myocardial infarction, reperfusion injury and acute/chronic inflammation

U.S. Patent Publication No. 2005/024552 describes pyridinone and pyrimidinone derivatives as Lp-PLA₂ inhibitors and their use in treatment of atherosclerosis, diabetes, angina pectoris, after ischaemia, reperfusion and psoriasis.

U.S. Patent Publication No. 2006/0241126 describes pyrimidinone derivatives as Lp-PLA₂ inhibitors and their use in treatment of atherosclerosis, diabetes, hypertension, angina pectoris, after ischaemia reperfusion and psoriasis.

U.S. Patent Publication No. 2008/0090851 describes bicyclic heteroaromatic compounds as Lp-PLA₂ inhibitors and their use in treatment of atherosclerosis, diabetes, rheumatoid arthritis, stroke, myocardial infarction, reperfusion injury and acute/chronic inflammation

U.S. Patent Publication No. 2010/0144765 describes 5,6-trimethylenepyrimidin-4-ones as Lp-PLA₂ inhibitors and their use in treatment of atherosclerosis, diabetes, rheumatoid arthritis, stroke, myocardial infarction, reperfusion injury, acute/chronic inflammation and Alzheimer's disease.

### SUMMARY OF THE INVENTION

The present disclosure relates to novel indolizine derivatives useful as inhibitors of lipoprotein associated phospholipase A2 (Lp-PLA2) and/or 15-lipoxygenase (15-LOX). In some embodiments, the indolizine derivatives are useful as dual inhibitors of lipoprotein associated phospholipase A2 (Lp-PLA2) and 15-lipoxygenase (15-LOX).

Indolizine has the structure and ring atom numbering shown below and forms the nucleus of the derivatives disclosed herein:

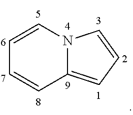

In one aspect, the present disclosure provides indolizine derivatives of Formula I:

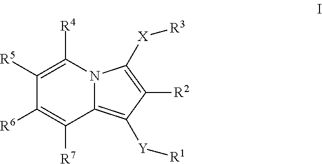

and pharmaceutically acceptable salts thereof, wherein

X and Y are independently C(O), C(S), NH, NR^{a}, S, O, where Y can be present or absent;

R¹ is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO2, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

with the proviso that when Y is C(O), R¹ is not C(O)NH₂, C(O)NHR^{d} or C(O)NR^{c}R^{d};

R² is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO2, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

R³ is a non-interfering substituent selected from C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}OC(O)NH₂, OC(O)NHR^{d}, OC(O)NR^{d}R^{d}, OC(O)OR^{d}, C(O)R^{e}, C(O)NH₂, C(O)NHR^{d}, C(O)NR^{d}R^{d}, C(O)OR^{d}, NH₂, NR^{f}H, NR^{f}R^{f}, NR^{e}C(O)NH₂, NR^{e}C(O)R^{d}, NR^{e}C(O)OR^{d} and NR^{e}C(O)NR^{e}R^{e},

wherein each C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}, OC(O)NHR^{d}, OC(O)NR^{d}R^{d}, OC(O)OR^{d}, C(O)R^{e}, C(O)NHR^{d}, C(O)NR^{d}R^{d}, C(O)OR^{d}, NR^{f}H, NR^{f}R^{f}, NR^{e}C(O)NH₂, NR^{e}C(O)R^{d}, NR^{e}C(O)OR^{d} and NR^{e}C(O)NR^{e}R^{e} is optionally substituted by one or more substituents independently selected from halogen, C₁₋₆ alkyl, C₁₋₆ haloalkyl and C₃-C₄ cycloalkyl;

with the proviso that when X is C(O), R³ is not C(O)NH₂, C(O)NHR^{d} or C(O)NR^{d}R^{d};

R⁴, R⁵, R⁶ and R⁷ are each non-interfering substituents independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH², NR^{b}C(O)R^{a}, NR^{b}C(O)OR^{a} and NR^{b}C(O)NR^{b}R^{b};

Ra is C₁₋₆ alkyl, C₁₋₆ haloalkyl, C₂₋₆ alkenyl, C₂₋₆ alkynyl, alkylchalcogen, arylchalcogen, aryl, cycloalkyl, heteroaryl, heterocycloalkyl, arylalkyl, heteroarylalkyl,

Rb is H, C₁₋₆ alkyl, C₁₋₆ haloalkyl, C₂₋₆ alkenyl, C₂₋₆ alkynyl, cyclopropyl, amino, alkylchalcogen, arylchalcogen, aryl, cycloalkyl, heteroaryl, heterocycloalkyl, arylalkyl, heteroarylalkyl;

Rc is C₁₋₆ alkyl, C₁₋₆ haloalkyl, C₂₋₆ alkenyl, C₂₋₆ alkynyl, alkylchalcogen, arylchalcogen, aryl, cycloalkyl, heteroaryl, heterocycloalkyl, arylalkyl, heteroarylalkyl;

Rd is C₁₋₆ alkyl, C₁₋₆ haloalkyl, C₂₋₆ alkenyl, C₂₋₆ alkynyl, C₃₋₄ cycloalkyl, amino;

Re is H, C₁₋₆ alkyl, C₁₋₆ haloalkyl, C₂₋₆ alkenyl, C₂₋₆ alkynyl, C3-4 cycloalkyl, cyclopropyl, amino; and

Rf is C₁₋₆ alkyl, C₁₋₆ haloalkyl, C₂₋₆ alkenyl, C₂₋₆ alkynyl, C3-4 cycloalkyl.

In some embodiments, X and Y are independently C(O) or C(S);
R1 is selected from C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, ORa and SRa,
wherein each of C1-6 alkyl, C1-6 haloalkyl, C2-6 alkenyl, C2-6 alkynyl, aryl, cycloalkyl, heteroaryl, heterocycloalkyl, arylalkyl, heteroarylalkyl, ORa and SRa is optionally substituted by one or more substituents independently selected from halogen, C1-6 alkyl, C1-6 haloalkyl, C2-6 alkenyl, C2-6 alkynyl, aryl, cycloalkyl, heteroaryl, heterocycloalkyl, arylalkyl, heteroarylalkyl, ORa and SRa;
R2 is selected from C1-6 alkyl, C1-6 haloalkyl, C2-6 alkenyl, C2-6 alkynyl, aryl, cycloalkyl, heteroaryl, heterocycloalkyl, arylalkyl, heteroarylalkyl, ORa and SRa;
wherein each of C1-6 alkyl, C1-6 haloalkyl, C2-6 alkenyl, C2-6 alkynyl, aryl, cycloalkyl, heteroaryl, heterocycloalkyl, arylalkyl, heteroarylalkyl, ORa or SRa, is optionally substituted by one or more substituents independently selected from halogen, C1-6 alkyl, C1-6 haloalkyl, C2-6 alkenyl, C2-6 alkynyl, aryl, cycloalkyl, heteroaryl, heterocycloalkyl, arylalkyl, heteroarylalkyl, ORa and SRa;
R3 is selected from C1-6 alkyl, C1-6 haloalkyl, C2-6 alkenyl, C2-6 alkynyl, C3-C4 cycloalkyl, ORd or SRd; and
R4, R5, R6, R7, Ra, Rb, Rc Rd, Re and Rf are as defined in claim 1

In some embodiments, X and Y are independently C(O) or C(S). In some embodiments, X and Y are C(O). In some embodiments, R³ is C₁-C₄ alkyl, such as methyl, ethyl, n-propyl, isopropyl, n-butyl or t-butyl.

In another aspect, the present disclosure provides a method of preparing an indolizine derivative as disclosed herein.

In another aspect, the present disclosure relates to a method of inhibiting an activity of Lp-PLA₂ and/or 15-LOX, comprising contacting Lp-PLA₂ and/or 15-LOX with a compound as disclosed herein.

In some embodiments, the present disclosure relates to a method of inhibiting an activity of Lp-PLA₂ and 15-LOX, comprising contacting Lp-PLA₂ and 15-LOX with a compound as disclosed herein.

In some embodiments, the present disclosure provides methods of treating a disease or condition associated with Lp-PLA₂ and/or 15-LOX by administering to a patient a therapeutically effective amount of a compound as disclosed herein.

In some embodiments, the present disclosure provides indolizine derivatives as disclosed herein, or pharmaceutically acceptable salts thereof, for use in therapy.

In some embodiments, the present disclosure provides indolizine derivatives as disclosed herein, or pharmaceutically acceptable salts thereof, for the manufacture/preparation of a medicament for use in therapy.

In some embodiments, the present disclosure provides pharmaceutical compositions comprising a compound as disclosed herein and at least one pharmaceutically acceptable carrier or diluent.

In some embodiments, kits comprising the compounds or compositions of the present disclosure are provided.

### DETAILED DESCRIPTION

In accordance with the present disclosure, there are provided indolizine derivatives of the general Formula I:

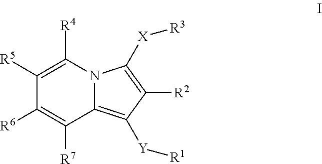

and pharmaceutically acceptable salts thereof, wherein

X and Y are independently C(O), C(S), NH, NR^{a}, S, O, where Y can be present or absent;

R¹ is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)ORa and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO2, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

with the proviso that when Y is C(O), R¹ is not C(O)NH₂, C(O)NHR^{d} or C(O)NR^{c}R^{d};

R² is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO2, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},

R³ is a non-interfering substituent selected from C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}OC(O)NH₂, OC(O)NHR^{d}, OC(O)NR^{d}R^{d}, OC(O)OR^{d}, C(O)R^{e}, C(O)NH₂, C(O)NHR^{d}, C(O)NR^{d}R^{d}, C(O)OR^{d}, NH₂, NR^{f}H, NR^{f}R^{f}, NR^{e}C(O)NH₂, NR^{e}C(O)R^{d}, NR^{e}C(O)OR^{d} and NR^{e}C(O)NR^{e}R^{e},

wherein each C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}, OC(O)NHR^{d}, OC(O)NR^{d}R^{d}, OC(O)OR^{d}, C(O)R^{e}, C(O)NHR^{d}, C(O)NR^{d}R^{d}, C(O)OR^{d}, NR^{f}H, NR^{f}R^{f}, NR^{e}C(O)NH₂, NR^{e}C(O)R^{d}, NR^{e}C(O)OR^{d} and NR^{e}C(O)NR^{e}R^{e} is optionally substituted by one or more substituents independently selected from halogen, C₁₋₆ alkyl, C₁₋₆ haloalkyl and C₃-C₄ cycloalkyl,

with the proviso that when X is C(O), R³ is not C(O)NH₂, C(O)NHR^{d} or C(O)NR^{d}R^{d};

R⁴, R⁵, R⁶ and R⁷ are each non-interfering substituents independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{f}H, NR^{c}R^{c}, NR^{b}C(O)NH₂, NR^{b}C(O)R^{a}, NR^{b}C(O)OR^{a} and NR^{b}C(O)NR^{b}R^{b},

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH², NR^{b}C(O)R^{a}, NR^{b}C(O)OR^{a} and NR^{b}C(O)NR^{b}R^{b};

R^{a} is C₁₋₆ alkyl, C₁₋₆ haloalkyl, C₂₋₆ alkenyl, C₂₋₆ alkynyl, alkylchalcogen, arylchalcogen, aryl, cycloalkyl, heteroaryl, heterocycloalkyl, arylalkyl, heteroarylalkyl,

R^{b} is H, C₁₋₆ alkyl, C₁₋₆ haloalkyl, C₂₋₆ alkenyl, C₂₋₆ alkynyl, cyclopropyl, amino, alkylchalcogen, arylchalcogen, aryl, cycloalkyl, heteroaryl, heterocycloalkyl, arylalkyl, heteroarylalkyl;

R^{c} is C₁₋₆ alkyl, C₁₋₆ haloalkyl, C₂₋₆ alkenyl, C₂₋₆ alkynyl, alkylchalcogen, arylchalcogen, aryl, cycloalkyl, heteroaryl, heterocycloalkyl, arylalkyl, heteroarylalkyl;

R^{d} is C₁₋₆ alkyl, C₁₋₆ haloalkyl, C₂₋₆ alkenyl, C₂₋₆ alkynyl, C₃₋₄ cycloalkyl, amino;

R^{e} is H, C₁₋₆ alkyl, C₁₋₆ haloalkyl, C₂₋₆ alkenyl, C₂₋₆ alkynyl, C₃₋₄ cycloalkyl, cyclopropyl, amino; and

R^{f} is C₁₋₆ alkyl, C₁₋₆ haloalkyl, C₂₋₆ alkenyl, C₂₋₆ alkynyl, C₃₋₄ cycloalkyl.

In some embodiments, there is provided a compound of Formula I wherein:
X and Y are independently C(O) or C(S);
R¹ is selected from C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a}, wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a} is optionally substituted by one or more substituents independently selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};
R² is selected from C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} or SR^{a}, is optionally substituted by one or more substituents independently selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};
R³ is selected from C₁₋₆ alkyl, C₁₋₆ haloalkyl, C₂₋₆ alkenyl, C₂₋₆ alkynyl, C₃-C₄ cycloalkyl, OR^{d} or SR^{d}; and
R⁴, R⁵, R⁶, R⁷, R^{a}, R^{b}, R^{c} R^{d}, R^{e} and R^{f} are as defined above.

In some embodiments, X and Y are independently C(O) or C(S);
R¹ is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, C₅ heteroaryl;
R² is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, C₅ heteroaryl;
R³ is selected from C₁₋₄ alkyl, C₁₋₄ haloalkyl, C₃-C₄ cycloalkyl, OR^{d} or SR^{d}; wherein R^{d} is C₁₋₄ alkyl, C₁₋₄ haloalkyl, C₃-C₄ cycloalkyl;
R⁴, R⁵, R⁶ and R⁷ are independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, alkylchalcogen, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}; and
R^{a}, R^{b} R^{c} and R^{d} are as defined above.

In embodiments of Formula I where Y is absent, R¹ attaches directly to the indolizine ring.

In some embodiments of Formula I, X and Y are independently C(O) or C(S). In some embodiments, X and Y are independently C(O) or C(S). In some, embodiments, X is C(O). In some embodiments, Y is C(O). In some, embodiments, X and Y are C(O).

In some embodiments, R¹ is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, C₅ heteroaryl.

In some embodiments, R² is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, C₅ heteroaryl.

In some embodiments, wherein R³ is selected from C₁₋₄ alkyl, C₁₋₄ haloalkyl, C₃-C₄ cycloalkyl, OR^{d} or SR^{d}; wherein R^{d} is C₁₋₄ alkyl, C₁₋₄ haloalkyl, C₃-C₄ cycloalkyl.

In some embodiments, R⁴, R⁵, R⁶ and R⁷ are independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, alkylchalcogen, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}; and
R^{a}, R^{b} R^{c} and R^{d} are as defined above.

In some embodiments, X and Y are independently C(O) or C(S); R¹ and R² are independently selected from C₁₋₆ alkyl, C₆-C₁₀ aryl, and C₅-C₁₂ heteroaryl, wherein each is optionally substituted by one or more substituents independently selected from halogen, C₁₋₄ alkyl, C₁₋₄ haloalkyl, OR^{a} and SR^{a}; where R^{a} is C₁₋₄ alkyl, or C₁₋₄ haloalkyl; and R³ is C₁₋₄ alkyl.

In some embodiments, R¹ and R² are each C₆ aryl, each said aryl being independently substituted with 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

In some embodiments, R¹ and/or R² is C₆ aryl substituted with —OMe or —SMe.

In some embodiments, where R¹ and/or R² is substituted C₆ aryl, each said ring substituent is in a para or meta position on the ring.

In some embodiments, R⁴, R⁵, R⁶ and R⁷ are independently selected from H, OH, halogen, CN, NO₂, C₁₋₄ alkyl and C₁₋₄ haloalkyl.

In some embodiments, one or both of R⁴ and R⁵ are halogen, OH, CN, NO₂, C₁₋₄ alkyl and C₁₋₄ haloalkyl

In some embodiments, R³ is methyl.

In some embodiments, there are provided compounds of Formula II:

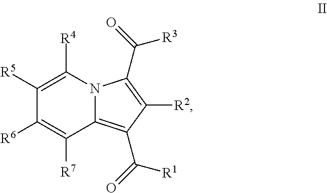

or a pharmaceutically acceptable salt thereof, wherein R¹-R⁷ are as defined herein.

In some embodiments, there are provided compounds of Formula III:

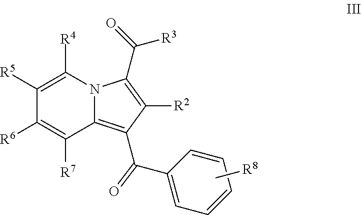

or a pharmaceutically acceptable salt thereof wherein R²-R⁷ are as defined herein.

In some embodiments, R⁸ represents 1, 2 or 3 non-interefering reing substituents selected from halogen, OH, NO₂, optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{b}C(O)NH₂, NR^{b}C(O)R^{a}, NR^{b}C(O)OR^{a} and NR^{b}C(O)NR^{b}R^{b}.

In some embodiments, there are provided compounds of Formula IV:

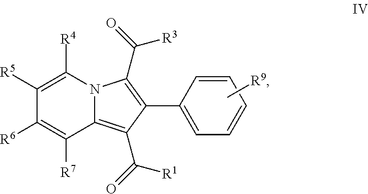

or a pharmaceutically acceptable salt thereof, wherein R¹, R³, R⁴-R⁷ are as defined herein.

In some embodiments, R⁹ represents 1, 2 or 3 non-interefering ring substituents selected from halogen, OH, NO₂, optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{b}C(O)NH₂, NR^{b}C(O)R^{a}, NR^{b}C(O)OR^{a} and NR^{b}C(O)NR^{b}R^{b}.

In some embodiments, there are provided compounds of Formula V:

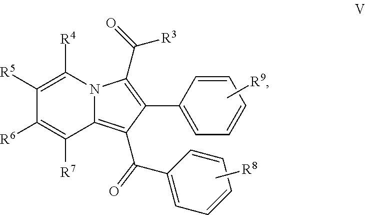

or a pharmaceutically acceptable salt thereof, wherein R³, R⁴-R⁷ are as defined herein.

In some embodiments, R⁸ and R⁹ independently represent 1, 2 or 3 non-interefering ring substituents selected from halogen, OH, NO₂, optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}R^{c}, NR^{b}C(O)NH₂, NR^{b}C(O)R^{a}, NR^{b}C(O)OR^{a} and NR^{b}C(O)NR^{b}R^{b}.

In some embodiments, there are provided compounds of Formula VI:

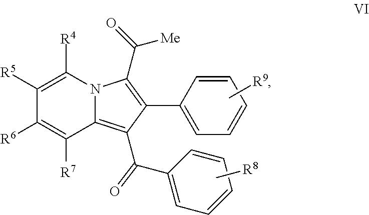

or a pharmaceutically acceptable salt thereof, wherein R¹-R⁷ are as defined herein.

In some embodiments, R⁸ and R⁹ independently represent 1, 2 or 3 non-interefering ring substituents selected from halogen, OH, NO₂, optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}R^{c}, NR^{b}C(O)NH₂, NR^{b}C(O)R^{a}, NR^{b}C(O)OR^{a} and NR^{b}C(O)NR^{b}R^{b}.

In some embodiments, wherein R⁸ and R⁹ are independently 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

In some embodiments, each said ring substituent is in a para or meta position on the ring.

In some embodiments, R⁴, R⁵, R⁶ and R⁷ are independently selected from H, OH, halogen, CN, NO₂, C₁₋₄alkyl and C₁₋₄haloalkyl.

In some embodiments, R⁴, R⁵, R⁶ and R⁷ are each H.

In some embodiments, there are provided compounds of Formula VII:

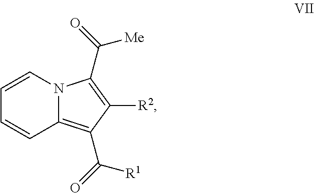

or a pharmaceutically acceptable salt thereof, wherein R¹ and R² are as defined herein.

In some embodiments of the compounds of Formula I, II, IV or VII, R¹ is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄alkyl, C₁₋₄haloalkyl, C₆ aryl, C₃-C₄ cycloalkyl, C₅ heteroaryl.

In some embodiments, R¹ is C₆ aryl substituted with 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

In some embodiments of the compounds of Formula I, II, III or VII, R² is selected from C₁₋₆ alkyl, C₁₋₆ haloalkyl, C₆-C₁₀ aryl, C₃-C₆ cycloalkyl, C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄alkyl, C₁₋₄haloalkyl, C₆ aryl, C₃-C₄ cycloalkyl, C₅ heteroaryl.

In some embodiments, R² is C₆ aryl substituted with 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

In some embodiments of the compounds disclosed herein, R¹ and/or R² are —OMe or —SMe.

In some embodiments of the compounds disclosed herein, each said ring substituent R8 or R9 is in a para or meta position.

In some embodiments of the compounds of Formula I, II, III, IV or V,

The compound of Formula I according to claim 1, 2 or 3, Formula II of claim 7, formula III of claim 8, Formula IV of claim 9, or Formula V of claim 10, wherein R³ is selected from C₁₋₄ alkyl, C₁₋₄ haloalkyl, C₃-C₄ cycloalkyl, OR^{d} or SR^{d}; wherein R^{d} is C₁₋₄ alkyl, C₁₋₄ haloalkyl, C₃-C₄ cycloalkyl.

In some embodiments, R³ is selected from C₁₋₄ alkyl, C₁₋₄ haloalkyl, C₃-C₄ cycloalkyl, OR^{d} or SR^{d}; wherein R^{d} is C₁₋₄ alkyl, C₁₋₄ haloalkyl, C₃-C₄ cycloalkyl. In some embodiments, R³ is C₁₋₄ alkyl. In some embodiments, R³ is methyl.

In some embodiments, R⁴, R⁵, R⁶ and R⁷ are independently selected from H, OH, halogen, CN, NO₂, C₁₋₄ alkyl and C₁₋₄ haloalkyl. In some embodiments, one or both of R⁴ and R⁵ are halogen, OH, CN, NO₂, C₁₋₄ alkyl or C₁₋₄ haloalkyl. In some embodiments, R⁴, R⁵, R⁶ and R⁷ are each H.

In some embodiments, there is provided a compound of Formula I wherein: X and Y are independently C(O) or C(S); R¹ and R² are independently selected from C₁₋₆ alkyl, C₆-C₁₀ aryl, and C₅-C₁₂ heteroaryl, wherein each is optionally substituted by one or more substituents independently selected from halogen, C₁₋₄ alkyl, C₁₋₄ haloalkyl, OR^{a} and SR^{a}; where IV is C₁₋₄ alkyl, or C₁₋₄ haloalkyl; R³ is C₁₋₄ alkyl; and R⁴-R⁷ are each H.

In some embodiments, there is provided a compound of Formula I wherein: X and Y are C(O); R¹ and R² are independently selected from C₁₋₆ alkyl, C₆ aryl, wherein each is optionally substituted by one or more substituents independently selected from halogen, C₁₋₄ alkyl, C₁₋₄ haloalkyl, OMe, OEt, SMe oe SEt; R³ is methyl; and R⁴-R⁷ are each H.

In some embodiments, haloalkyl is CF³.

In some embodiments, there is provided a compound of Formula VI:

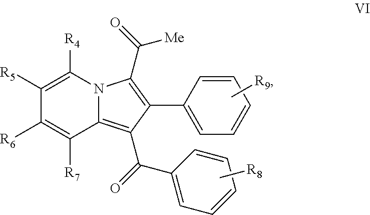

or a pharmaceutically acceptable salt thereof, wherein R⁴, R⁵, R⁶ and R⁷ are independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, alkylchalcogen, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b};

wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}; and

R⁸ and R⁹ each represent 1, 2 or 3 non-interfering ring substituents being independently selected from H, halogen, OH, C₁₋₆ alkyl, C₁₋₆ haloalkyl, C₆-C₁₀ aryl, C₃-C₆ cycloalkyl, C₅-C₁₂ heteroaryl, wherein each of C₁₋₆ alkyl, C₁₋₆ haloalkyl, C₆-C₁₀ aryl, C₃-C₆ cycloalkyl, C₅-C₁₂ heteroaryl is optionally substituted by one or more substituents independently selected from halogen, OH, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄haloalkyl, C₆ aryl, C₃-C₄ cycloalkyl, C₅ heteroaryl.

In some embodiments, R⁸ and R⁹ each represent 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

In some embodiments, wherein at least one of R⁸ and R⁹ is halogen. In some embodiments, halogen is Cl, Br or F.

In some embodiments, at least one of R⁸ and R⁹ is CF₃. In some embodiments, at least one of R⁸ and R⁹ is OMe, Oet, SMe, or SEt. In some embodiments, at least one of R⁸ and R⁹ is methyl, ethyl, n-propyl, isopropyl, n-butyl, t-butyl. In some embodiments, R⁸ and R⁹, for each occurrence, are in the meta or para position.

In some embodiments, the compound of Formula I is a compound having the following structure:

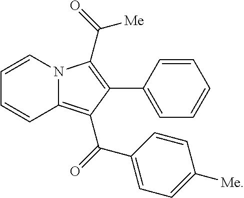 [SMILES: CC(=O)c1c(-c2ccccc2)c(C(=O)c2ccc(C)cc2)c2ccccn12]

In some embodiments, the compound of Formula I is a compound having the following structure:

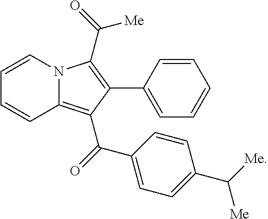 [SMILES: CC(=O)c1c(-c2ccccc2)c(C(=O)c2ccc(C(C)C)cc2)c2ccccn12]

In some embodiments, the compound of Formula I is a compound having the following structure:

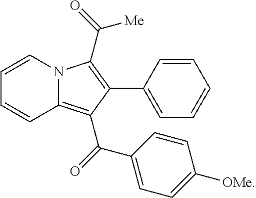 [SMILES: COc1ccc(C(=O)c2c(-c3ccccc3)c(C(C)=O)n3ccccc23)cc1]

In some embodiments, the compound of Formula I is a compound having the following structure:

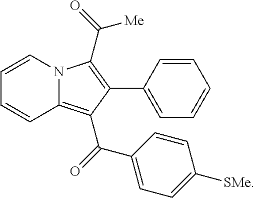 [SMILES: CSc1ccc(C(=O)c2c(-c3ccccc3)c(C(C)=O)n3ccccc23)cc1]

In some embodiments, the compound of Formula I is a compound having the following structure:

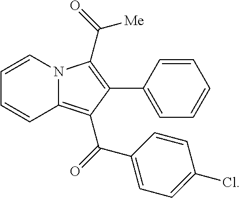 [SMILES: CC(=O)c1c(-c2ccccc2)c(C(=O)c2ccc(Cl)cc2)c2ccccn12]

In some embodiments, the compound of Formula I is a compound having the following structure:

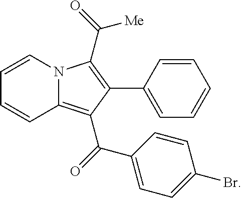 [SMILES: CC(=O)c1c(-c2ccccc2)c(C(=O)c2ccc(Br)cc2)c2ccccn12]

In some embodiments, the compound of Formula I is a compound having the following structure:

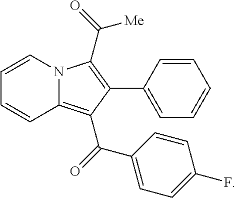 [SMILES: CC(=O)c1c(-c2ccccc2)c(C(=O)c2ccc(F)cc2)c2ccccn12]

In some embodiments, the compound of Formula I is a compound having the following structure:

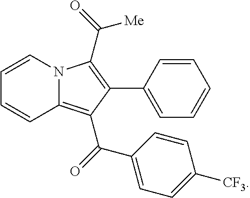 [SMILES: CC(=O)c1c(-c2ccccc2)c(C(=O)c2ccc(C(F)(F)F)cc2)c2ccccn12]

In some embodiments, the compound of Formula I is a compound having the following structure:

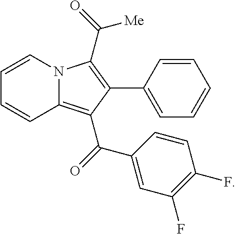 [SMILES: CC(=O)c1c(-c2ccccc2)c(C(=O)c2ccc(F)c(F)c2)c2ccccn12]

In some embodiments, the compound of Formula I is a compound having the following structure:

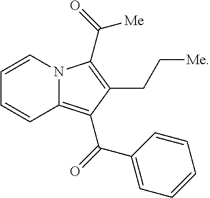 [SMILES: CCCc1c(C(=O)c2ccccc2)c2ccccn2c1C(C)=O]

In some embodiments, the compound of Formula I is a compound having the following structure:

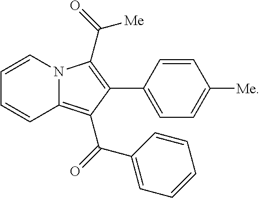 [SMILES: CC(=O)c1c(-c2ccc(C)cc2)c(C(=O)c2ccccc2)c2ccccn12]

In some embodiments, the compound of Formula I is a compound having the following structure:

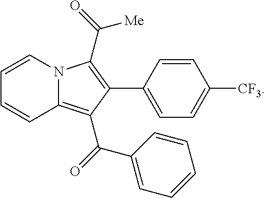 [SMILES: CC(=O)c1c(-c2ccc(C(F)(F)F)cc2)c(C(=O)c2ccccc2)c2ccccn12]

In some embodiments, the compound of Formula I is a compound having the following structure:

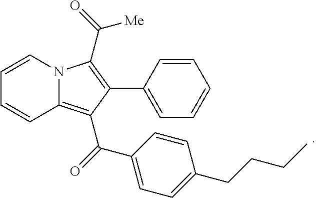 [SMILES: CCCCc1ccc(C(=O)c2c(-c3ccccc3)c(C(C)=O)n3ccccc23)cc1]

### DEFINITIONS

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl, unbranched or branched.

For compounds of the invention in which a variable appears more than once, each variable can be a different moiety selected from the Markush group defining the variable. For example, each of R¹, R², R³, R⁴, R⁵, R⁶, R⁷ and R⁸ can be a different moiety selected from the Markush group defining the variable. For another example, where a structure is described as encompassing multiple R groups that are simultaneously present on the same compound; the two R groups can represent different moieties selected from the Markush group defined for R. In another example, when an optionally multiple substituent is designated, such as a floating substituent on an aryl ring (e.g. R⁸ and R⁹), it is understood that substituent R can occur one or more times on the ring, and R can be a different moiety at each occurrence.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the singular term “a” includes the plural “at least one”.

As used herein, the term “compound” may in include pharmaceutically acceptable salt and ester forms where appropriate. A skilled person will understand how to prepare the compounds as salts and esters using routine methods known in the chemical arts.

The term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl group.

As used herein, the term “alkyl” is meant to refer to a saturated hydrocarbon group which is straight-chained or branched. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, from 1 to about 3 carbon atoms, from 1 to 2 carbon atoms, or 1 carbon atom. For example, alkyl may include C₁-C₂₀, C₂-C₂₀, C₁-C₁₀, C₁-C₈, C₁-C₆, C₁-C₄, C₁-C₃, or C₁-C₂ alkyl.

As used herein, “alkenyl” refers to an alkyl group having one or more double carbon-carbon bonds. Example alkenyl groups include, but are not limited to, ethenyl, propenyl, cyclohexenyl, and the like.

As used herein, “alkynyl” refers to an alkyl group having one or more triple carbon-carbon bonds. Example alkynyl groups include, but are not limited to, ethynyl, propynyl, and the like.

As used herein, “haloalkyl” refers to an alkyl group having one or more halogen substituents. Example haloalkyl groups include, but are not limited to CF3, C₂F₅, CHF₂, CCl₃, CHCl₂, C₂Cl₅, CH₂CF₃, and the like.

As used herein, the term “chalcogen” refers to a Group 16 element of the Periodic Table, in particular, O, N or S.

As used herein, the term “alkylchalcogen” refers to an alkyl group coupled to a chalcogen, in particular, an —O-alkyl, —S-alkyl or —N-alkyl group. Examples of alkylchalcogen include alkoxy, alkylthio and alkylamino (C₁-C₆ alkyl)chalcogen refers to chalcogen coupled to C₁-C₆ alkyl.

As used herein, the term “alkoxy” refers to an —O-alkyl group where alkyl is straight-chained or branched. Examples of alkoxy groups include, but are not limited to, methoxy (OMe), ethoxy (OEt), propoxy (e.g., n-propoxy and isopropoxy), butoxy (e.g., n-butoxy, isobutoxy, t-butoxy), pentoxy (e.g., n-pentoxy, isopentoxy, neopentoxy), and the like.

As used herein, the term “alkylthio” refers to an —S-alkyl group where alkyl is straight-chained or branched. Examples of alkylthio groups include, but are not limited to, methylthio (SMe), ethylthio (SEt), propylthio (e.g., n-propylthio and isopropylthio), butylthio (e.g., n-butylthio, isobutylhio, t-butylthio), pentylthio (e.g., n-pentylthio, isopentylthio, neopentylthio), and the like.

As used herein, the term “alkylamino” refers to an N-alkyl group where alkyl is straight-chained or branched. Examples of alkylamino groups include, but are not limited to, methylamino (NHMe), ethylamino (NHEt), propylamino (e.g., n-propylamino and isopropylamino), butylamino (e.g., n-butylamino, isobutyamino, t-butylamino), pentylamino (e.g., n-pentylamino, isopentylamino, neopentylamino), and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms. In some embodiments, aryl groups have from 6 to about 10 carbon atoms. In some embodiments, aryl groups have 5 or 6 carbon atoms.

As used herein, “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic (e.g., having 2, 3 or 4 fused rings) ring systems as well as spiro ring systems. A cycloalkyl group can contain from 3 to about 15, from 3 to about 10, from 3 to about 8, from 3 to about 6, from 4 to about 6, from 3 to about 5, or from 5 to about 6 ring-forming carbon atoms. Ring-forming carbon atoms of a cycloalkyl group can be optionally substituted by oxo or sulfido. Example cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, “heteroaryl” groups refer to an aromatic heterocycle having up to 20 ring-forming atoms and having at least one heteroatom ring member (ring-forming atom) such as sulfur, oxygen, or nitrogen. In some embodiments, the heteroaryl group has at least one, or one or more, heteroatom ring-forming atoms each independently selected from sulfur, oxygen, and nitrogen. Heteroaryl groups include monocyclic and polycyclic (e.g., having 2, 3 or 4 fused rings) systems. Examples of heteroaryl groups include without limitation, indolizinyl, indolinyl, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, and the like. In some embodiments, the heteroaryl group has from 1 to about 20 carbon atoms, and in further embodiments from about 1 to about 5, from about 1 to about 4, from about 1 to about 3, from about 1 to about 2, carbon atoms as ring-forming atoms. In some embodiments, the heteroaryl group contains 3 to about 14, 3 to about 7, or 5 to 6 ring-forming atoms. In some embodiments, the heteroaryl group contains 5 ring-forming atoms. In some embodiments, the heteroaryl group contains 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “heterocycloalkyl” refers to non-aromatic heterocycles having up to 20 ring-forming atoms including cyclized alkyl, alkenyl, and alkynyl groups where one or more of the ring-forming carbon atoms is replaced by a heteroatom such as an O, N, or S atom. Heterocycloalkyl groups can be mono or polycyclic (e.g., both fused and spiro systems). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. Ring-forming carbon atoms and heteroatoms of a heterocycloalkyl group can be optionally substituted by oxo or sulfido. For example, a ring-forming S atom can be substituted by 1 or 2 oxo [i.e., form a S(O) or S(O)₂]. For another example, a ring-forming C atom can be substituted by oxo (i.e., form carbonyl). Also included in the definition of heterocycloalkyl are moieties that have one or more aromatic rings fused (i.e., having a bond in common with) to the nonaromatic heterocyclic ring, for example pyridinyl, thiophenyl, phthalimidyl, naphthalimidyl, and benzo derivatives of heterocycles such as indolene, isoindolene, isoindolin-1-one-3-yl, 4,5,6,7-tetrahydrothieno[2,3-c]pyridine-5-yl, 5,6-dihydrothieno[2,3-c]pyridin-7(4H)-one-5-yl, and 3,4-dihydroisoquinolin-1(2H)-one-3yl groups. Ring-forming carbon atoms and heteroatoms of the heterocycloalkyl group can be optionally substituted by oxo or sulfido. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo. In some embodiments, it refers to fluoro, chloro, and bromo.

As used herein “halide” refers to Cl⁻, Br⁻ or I⁻.

As used herein, “haloalkoxy” refers to an O-haloalkyl group. An example haloalkoxy group is OCF₃.

As used herein, “arylalkyl” refers to a C₁₋₆ alkyl substituted by aryl and “cycloalkylalkyl” refers to C₁₋₆ alkyl substituted by cycloalkyl.

As used herein, “heteroarylalkyl” refers to a C₁₋₆ alkyl group substituted by a heteroaryl group, and “heterocycloalkylalkyl” refers to a C₁₋₆ alkyl substituted by heterocycloalkyl.

As used here, “C(O)” refers to C(═O).

As used here, “C(S)” refers to C(═S).

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group (i.e., CH₃) is optionally substituted, then up to 3 hydrogen atoms on the carbon atom can be replaced with substituent groups. Optional substituents may include, for example, one or more substituents independently selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, where IV is C₁₋₆ alkyl, C₁₋₆ haloalkyl, C₂₋₆ alkenyl, C₂₋₆ alkynyl, C₃₋₄ cycloalkyl, amino; R^{b} is H, C₁₋₆ alkyl, C₁₋₆ haloalkyl, C₂₋₆ alkenyl, C₂₋₆ alkynyl, C₃₋₄ cycloalkyl, cyclopropyl, amino; and R^{c} is C₁₋₆ alkyl, C₁₋₆ haloalkyl, C₂₋₆ alkenyl, C₂₋₆ alkynyl, C₃₋₄ cycloalkyl. In some embodiments, optional substituents may also include one or more of OH, NO₂ and CN.

It is preferable that strong acid groups are not present at positions R¹ and/or R³. In some embodiments, compounds of Formula I do not contain an acid group at R³ and R¹. As used herein, “strong acid group” refers, in particular, to C(O)OH, SO₃H. Without wishing to be bound by theory, it is believed that avoiding strong acid groups at positions R¹ and R³, in particular, R³, results in increased selectivity for Lp-PLA₂, e.g. relative to sPLA₂ and COX. Acidic groups are present in several cyclooxygenase (COX) inhibitors, such as aspirin, indomethacin and ibuprofen. In some embodiments, compounds of Formula I contain non-polar groups at R³ and R¹, such as alkyl groups. In some embodiments, the compounds of the present disclosure do not contain selective COX-2 pharmacophores such as sulfomamide (—SO₂NH₂) or sulfonylmethyl (—SO₂Me). In some embodiments, the compounds disclosed herein do not contain SO₂ groups.

By “one or more substituents”, it is generally meant that 1, 2, 3, 4, or 5 substituents are present on a radical. A skilled person will be able to determine which substituents are preferred, and how many, depending on the compound. The substituents selected are preferably non-interfereing substituents. As used herein, the term, “non-interfering substitutent” refers to a substituent that does not prevent or significantly reduce the ability of the compounds to inhibit Lp-PLA2 and/or 15-LOX enzyme.

In some embodiment, when R2 and/or R3 are aryl, any substituent is in the para or meta position on the ring.

Some compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are encompassed and may be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated. A skilled person can readily determine whether a particular stereoisomer is preferred, e.g., for optimal enzyme inhibition, stability, or the like.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Some compounds of the present disclosure may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Some compounds of the present disclosure further include hydrates and solvates, as well as anhydrous and non-solvated forms.

The term, “compound,” as used herein is meant to include all stereoisomers, geometric iosomers, tautomers, and isotopes of the structures depicted.

All compounds and pharmaceutically acceptable salts thereof, can be prepared or present together with other substances such as water and solvents (e.g. hydrates and solvates) or can be isolated.

Compounds of the present disclosure can also include all isotopes of atoms occurring in the intermediates or final compounds. Isotopes include those atoms having the same atomic number but different mass numbers. For example, isotopes of hydrogen include tritium and deuterium.

In some embodiments, the compounds of the present disclosure, or salts thereof, are substantially isolated. By “substantially isolated” is meant that the compound is at least partially or substantially separated from the environment in which it is formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound or salt thereof. Methods for isolating compounds and their salts are routine in the art.

In some embodiments, compounds of the present disclosure are intended to include compounds with stable structures. As used herein, “stable compound” and “stable structure” are meant to indicate a compound that is sufficiently robust to survive isolation to a useful degree of purity from a reaction mixture, and formulation into an efficacious therapeutic agent.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g. a reaction temperature, that is about the temperature of the room in which the reaction is carried out, for example, a temperature from about 18° C. to about 30° C., typically, from about 20° C. to about 25° C.

In some embodiments, the present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

In some embodiments, the present invention also includes quaternary ammonium salts of the compounds described herein, where the compounds are primary amines, secondary amines, or tertiary amines. As used herein, “quaternary ammonium salts” refers to derivatives of the disclosed primary amine, secondary amine, or tertiary amine compounds wherein the parent amine compounds are modified by converting the amines to quaternary ammonium cations via alkylation (and the cations are balanced by anions such as Cl⁻, CH₃COO⁻, or CF₃COO⁻), for example methylation or ethylation.

### Synthesis Methods

In some embodiments, compounds of the present disclosure, including salts thereof, are prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC).

In some embodiments, compounds of Formula 1 are prepared using a synthesis method as outlined in Scheme 1 below, where X and Y are independently C(O) or C(S) and wherein each of R¹-R⁷ are as defined herein.

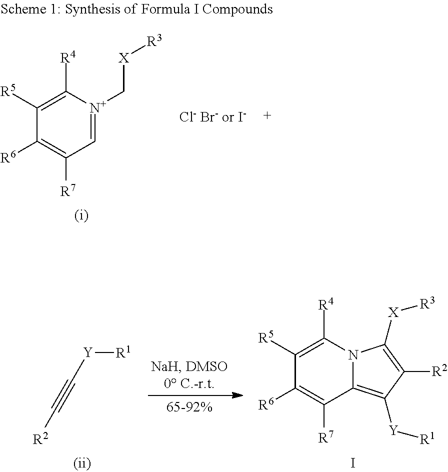

In some embodiments, compounds of Formula II are prepared using a novel synthesis method outlined in Scheme 2 below, wherein each of R¹-R⁷ are as defined herein.

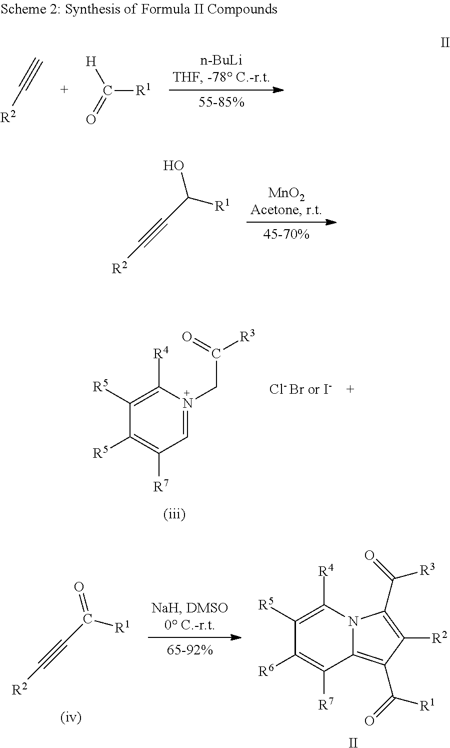

In some embodiments, compounds of select indolizine derivatives are prepared using a novel synthesis method outlined in Scheme 3 below, wherein R groups are as defined in Scheme 3.

### I

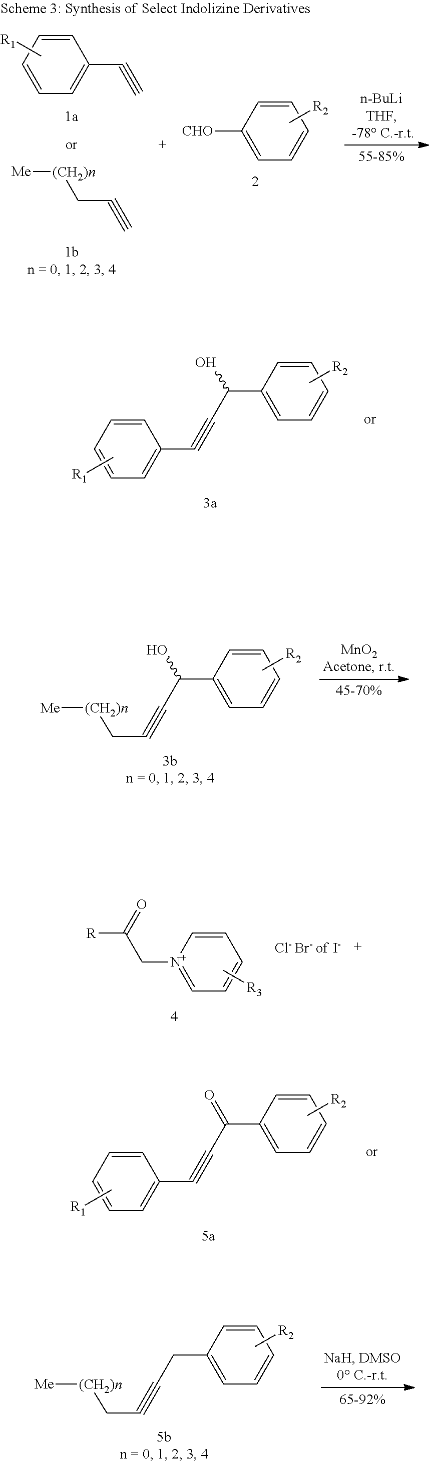

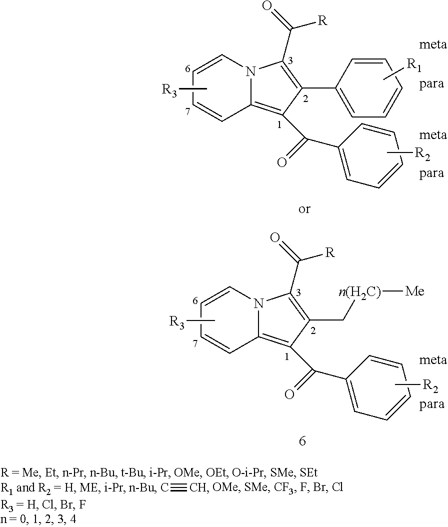

According to Scheme 3, to a solution of pyridinium ylide (3.84 mmol) and diphenylprop-2-yn-1-one (1.92 mmol) in 10-15 mL of DMSO at room temperature was added NaH (95%, 4.62 mmol) slowly with stirring and the reaction mixture was kept at room temperature for 1 h with stirring. Quenched with 10-15 mL of water and brine solution, washed with EtOAc (3×15 mL) and the combined organic layer was dried over anhydrous MgSO₄. The organic layer was evaporated in vacuo and the resulting residue was further purified by silica gel column chromatography using EtOAc:hexanes (1:3 or 1:2) or hexanes:acetone (7:1) respectively to afford indolizine derivatives (65-92%).

In some embodiments, there is provided a method of preparing a compound of Formula I, comprising reacting, in a solution of NaH in DMSO, a compound of formula (i)

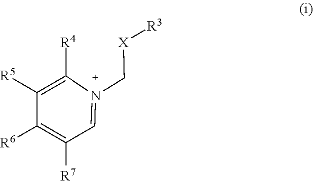

in the presence of a suitable ion, such as a halide, with a compound of formula (ii)

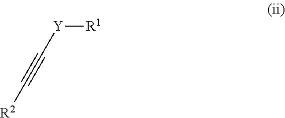

to form a compound of Formula I

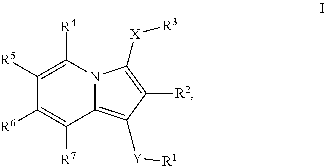

wherein X and Y are independently C(O) or C(S), and wherein each of R¹ to R⁷ are as defined herein.

In some embodiments, the halide is Cl⁻, Br⁻ or I⁻. In some embodiments, the halide is Cl⁻ or Br⁻. In some embodiments, the halide is Cl⁻. In some embodiments, the halide is Cl⁻ or Br⁻.

In some embodiments, the reaction employs a molar excess of NaH relative to (i). In some embodiments, the molar excess of NaH relative to (i) is about 1.1:1 to about 5:1, about 1.1:1 to about 3:1, about 1.1:1 to about 2:1, about 1.1:1 to about 1.5:1, or about 1.1:1, or about 1.2:1, or about 1.5:1.

In some embodiments, the reaction employs a molar excess of (i) relative to (ii). In some embodiments, the molar excess of (i) relative to (ii) is about 1.1:1 to about 5:1, about 1.1:1 to about 3:1, about 1.1:1 to about 2.5:1, about 1.1:1 to about 2:1, or about 1.5:1, or about 2:1, or about 3:1.

In some embodiments, the reaction employs a molar excess of (i) relative to (ii) of about 1.5:1 to about 2.5:1 and a molar excess of NaH relative to (i) of about 1.1:1 to about 1.5:1.

In some embodiments, the reaction employs a molar excess of (i) relative to (ii) of about 2:1 and a molar excess of NaH relative to (i) of about 1.2:1.

In some embodiments, the reaction takes place at a temperature of about 0° C. to about 60° C., about 10° C. to about 40° C., about 15° C. to about 30° C., about 20° C. to about 24° C., or at room temperature.

In some embodiments, the reaction is carried our while stirring for at least about 1 hour, about 1 hour to about 12 hours, about 1 hour to about 6 hours, about 1 hour to about 4 hours, about 1 hour to about 3 hours, about 1 hour to about 4 hours, or until substantially all of (i) has reacted.

In some embodiments, the reaction takes place at a temperature of about 15° C. to about 30° C. for about 1 hour to about 4 hours with a molar excess of NaH relative to (i) of between about 1.1.1 to about 1.5:1 and a molar excess of (i) relative to (ii) of about 1.5:1 to about 2.5:1.

In some embodiments a yield of about 65%-92% may be achieved.

In some embodiments, there is provided a method of preparing a compound of Formula II, comprising the steps of reacting, in a solution of NaH in DMSO, in the presence of halide, a compound of formula (iii)

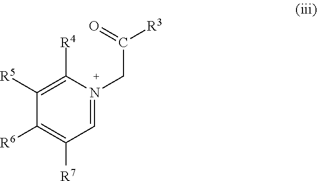

with a compound of formula (iv)

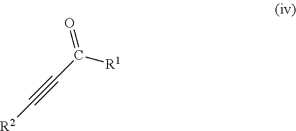

to form a compound of Formula II

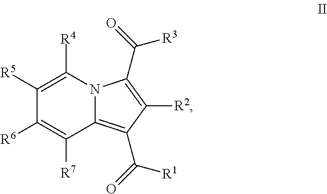

wherein each of R¹ to R⁷ are as defined herein.

In some embodiments, the halide is Cl⁻, Br⁻ or I⁻. In some embodiments, the halide is Cl⁻ or Br⁻. In some embodiments, the halide is Cl⁻. In some embodiments, the halide is Cl⁻ or Br⁻.

In some embodiments, the reaction employs a molar excess of NaH relative to (iii). In some embodiments, the molar excess of NaH relative to (iii) is about 1.1:1 to about 5:1, about 1.1:1 to about 3:1, about 1.1:1 to about 2:1, about 1.1:1 to about 1.5:1, or about 1.1:1, or about 1.2:1, or about 1.5:1.

In some embodiments, the reaction employs a molar excess of (iii) relative to (iv). In some embodiments, the molar excess of (iii) relative to (iv) is about 1.1:1 to about 5:1, about 1.1:1 to about 3:1, about 1.1:1 to about 2.5:1, about 1.1:1 to about 2:1, or about 1.5:1, or about 2:1, or about 3:1.

In some embodiments, the reaction employs a molar excess of (iii) relative to (iv) of about 1.5:1 to about 2.5:1 and a molar excess of NaH relative to (iii) of about 1.1:1 to about 1.5:1.

In some embodiments, the reaction employs a molar excess of (iii) relative to (iv) of about 2:1 and a molar excess of NaH relative to (iii) of about 1.2:1.

In some embodiments, the reaction takes place at a temperature of about 0° C. to about 60° C., about 10° C. to about 40° C., about 15° C. to about 30° C., about 20° C. to about 24° C., or at room temperature.

In some embodiments, the reaction is carried our while stirring for at least about 1 hour, about 1 hour to about 12 hours, about 1 hour to about 6 hours, about 1 hour to about 4 hours, about 1 hour to about 3 hours, about 1 hour to about 4 hours, or until substantially all of (iii) has reacted.

In some embodiments, the reaction takes place at a temperature of about 15° C. to about 30° C. for about 1 hour to about 4 hours with a molar excess of NaH relative to (iii) of between about 1.1.1 to about 1.5:1 and a molar excess of (iii) relative to (iv) of about 1.5:1 to about 2.5:1.

In some embodiments a yield of about 65%-92% may be achieved.

In some embodiments, the compound of formula (Iv) is obtained by

reacting a compound of formula (v)

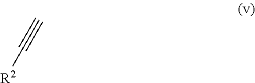

with a compound of formula (vi)

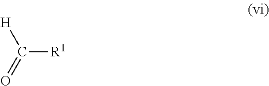

to form a compound of formula (vii)

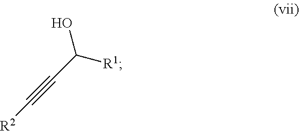

and

oxidizing the compound of formula (vii) to form a compound of formula (iv)

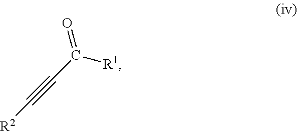

The step of reacting a compound of formula (v) with a compound of formula (vi) to form a compound of formula (vii) may be carried out according to methods known to those skilled in the art. The reaction may take place in a suitable solvent, such as an organic solvent. In some embodiments, the step of reacting a compound of formula (v) with a compound of formula (vi) to form a compound of formula (vii) is carried out in an organic solvent, such THF, in the presence of an organolithium reagent, such as n-BuLi. In some embodiments, the reaction takes place at −78° C. to room temperature. In some embodiments, the reaction takes place at room temperature. In some embodiments, a yield of 55-85% may be achieved.

The step of oxidizing a compound of (vii) to form a compound of formula (Iv) may be carried out according to methods known to those skilled in the art. Any suitable oxidizer may be used. In some embodiments, the oxidization step is carried out with MnO₂ as the oxidizer. In some embodiments, the oxidization step is carried out in acetone with MnO₂ as the oxidizer. In some embodiments, the oxidization takes place at room temperature. In some embodiments, a yield of 45-75% may be achieved.

Those skilled in the art can recognize that in all of the schemes described herein, if there are functional (reactive) groups present on a substituent group such as R¹, R², R³, R⁴, R⁵, R⁶, R⁷, R⁸, R⁹, etc., further modification can be made if appropriate and/or desired.

As used herein, the term “reacting” refers to the bringing together of designated chemical reactants such that a chemical transformation takes place generating a compound different from any initially introduced into the system. Reacting can take place in the presence or absence of solvent. A skilled person having regard to the present disclosure will be able to select appropriate conditions for a given reaction.

### Methods

In another aspect, there is provided a method of inhibiting Lₚ-PLA₂ and/or 15-LOX comprising contacting said Lₚ-PLA₂ and/or 15-LOX with a compound as described herein. In some embodiments, LP-PLA₂ and 15-LOX are inhibited

In some embodiments, the indolizine derivatives of the present disclosure have an IC₅₀ with respect to Lp-PLA₂ of less than about 1000 nM, 900 nM, 800 nM, 700 nM, 600 nM, 500 nM, 300 nM, 200 nM, 100 nM, 50 nM, 20 nM, 10 nM, 5 nM, 2 nM, or 1 nM. Accordingly, compounds of the present disclosure inhibit activity of Lp-PLA₂.

In some embodiments, compounds exhibiting an IC₅₀ with respect to Lp-PLA₂ of less than 1000 nM are preferred. In some embodiments, compounds exhibiting an IC₅₀ with respect to Lp-PLA₂ of less than 900 nM are preferred. In some embodiments, compounds exhibiting an IC₅₀ with respect to Lp-PLA₂ of less than 800 nM are preferred. In some embodiments, compounds exhibiting an IC₅₀ with respect to Lp-PLA₂ of less than 500 nM are preferred.

In some embodiments, the indolizine derivatives of the present disclosure have an IC₅₀ with respect to 15-LOX less than about 15 μM, 10 μM, 9 μM, 8 μM, 7 μM, 6 μM, 5 μM, 4 μM, 3 μM, 2 μM, or 1 μM. Accordingly, compounds of the present disclosure inhibit activity of 15-LOX.

In some embodiments, compounds exhibiting an IC₅₀ with respect to 15-LOX of less than 10 μM are preferred. In some embodiments, compounds exhibiting an IC₅₀ with respect to 15-LOX of less than 5 μM are preferred. In some embodiments, compounds exhibiting an IC₅₀ with respect to 15-LOX of less than 2.5 μM are preferred.

A skilled person can appreciate that an IC₅₀ of “less than” a given value is not intended to encompass impossible values such that no lower end of a range need been defined. However, in some cases, it may be desirable to specify a lower end of a range. In some embodiments, the indolizine derivatives of the present disclosure have an IC₅₀ with respect to Lp-PLA₂ of greater than about 0.001 nM. In some embodiments, the indolizine derivatives of the present disclosure have an IC₅₀ with respect to 15-LOX of greater than about 0.001 uM.

The term “inhibit” is meant to refer to an ability to decrease an activity of an enzyme. Thus, and “inhibitor” is a compound that inhibits an activity of an enzyme. In some embodiments, the inhibitor is a competitive inhibitor of binding at an enzyme binding site. In some embodiments, the inhibitor is a competitive non-covalent inhibitor. Enzyme inhibition assays are described in further detail in the Examples section. In some embodiments, the compounds of the present disclosure are dual inhibitors of Lp-PLA₂ of 15-LOX. In general, the compounds are more potent inhibitors of Lp-PLA₂ than 15-LOX. Although the potency differs, it is well know that synergies still be achieved by targeting two different enzymes with a single compound.

In some embodiments, the inhibition is selective for Lp-PLA₂ and/or 15-LOX over other enzymes, such as other forms of phospholipase enzyme, e.g. sPLA₂ IIA, or cyclooxygenase enzymes, e.g. COX-1 or COX-2. In some embodiments, the compounds used in the invention show 25% or more of binding to Lp-PLA₂ and/or 15-LOX comparing to other enzymes tested. In some embodiments, the IC₅₀ of compounds of the invention with respect to sPLA₂ IIA, COX-1 or COX-2, is greater than 10 μM, 20 μM, 50 μM, 100 μM, or 200 W. In some embodiments, the relative ratio of IC₅₀ of the compounds of invention with respect to sPLA₂ IIA, COX-1 or COX-2 to that with respect to Lp-PLA₂ and/or 15-LOX is greater than about 5:1, 10:1, 20:1, 50:1, 100:1, 200:1, 500:1, 1000:1, 2000:1, 5000:1, or 10000:1.

Accordingly, compounds of the invention can be used in methods of inhibiting Lp-PLA₂ and/or 15-LOX by contacting the Lp-PLA₂ and/or 15-LOX with a compound or compositions described herein. In some embodiments, compounds of the present invention can act as dual inhibitors of Lp-PLA₂ and 15-LOX. In further embodiments, the compounds of the invention can be used to inhibit activity of a Lp-PLA₂ and/or 15-LOX in an individual in need of inhibition of the enzyme by administering an inhibitory amount of a compound of the invention.

Another aspect of the present invention pertains to methods of treating an Lp-PLA₂-associated and/or 15-LOX-associated disease or condition in an individual (e.g., patient) by administering to the individual a therapeutically effective amount or dose of a compound of the present disclosure or a pharmaceutical composition thereof. In some embodiments, the individual has been diagnosed to have an Lp-PLA₂-associated and/or 15-LOX-associated disease or condition and is in need of treatment for the disease or condition. In some cases, the individual has been identified as being at risk of developing an Lp-PLA₂-associated and/or 15-LOX-associated disease or condition and is in need of preventative treatment. An Lp-PLA₂-associated and/or 15-LOX-associated disease can include any disease or condition that is directly or indirectly linked to elevated expression or activity of Lp-PLA₂ and/or 15-LOX, including increased expression and/or increased activity levels. The term “increased” is in relation to an individual that does not have the disease or condition associated with Lp-PLA₂ and 15-LOX. An Lp-PLA₂-associated and/or 15-LOX-associated disease can also include any disease or condition that can be prevented, ameliorated, or cured by inhibiting Lp-PLA₂ and/or 15-LOX.

Examples of 15-LOX-associated diseases or conditions include, but are not limited to, cardiovascular, inflammatory and proliferative disorders. Exemplary cardiovascular diseases and conditions associated with 15-LOX include atherosclerosis, stroke, myocardial infarction, acute coronary syndrome, coronary heart disease and peripheral arterial disease. Exemplary inflammatory diseases and conditions associated with 15-LOX include chronic/acute inflammation, rheumatoid arthritis, and asthma. Exemplary proliferative diseases and conditions associated with 15-LOX include cancers, such as prostate, pancreatic and colorectal cancers.

Examples of diseases or conditions associated with Lp-PLA₂ and 15-LOX include, but are not limited to, cardiovascular, inflammatory and proliferative disorders. Exemplary cardiovascular diseases and conditions associated with Lp-PLA₂ and 15-LOX include, but are not limited to, atherosclerosis, myocardial infarction, acute coronary syndrome, coronary heart disease, peripheral arterial disease, stroke, myocardial infarction, and reperfusion injury. Exemplary inflammatory diseases and conditions associated with Lp-PLA₂ and 15-LOX include acute/chronic inflammation, asthma, rheumatoid arthritis and psoriasis. Exemplary proliferative diseases and conditions associated with Lp-PLA₂ and 15-LOX include cancers, such as prostate, pancreatic and colorectal cancers. Another exemplary disease associated Lp-PLA₂ and 15-LOX is diabetes.

As used herein, the term “treating” or “treatment” refers to one or more of (1) preventing the disease or condition; for example, preventing a disease or condition in an individual who may be predisposed to the disease or condition but does not yet experience or display the pathology or symptomatology of the disease; (2) inhibiting or retarding disease progression; for example, inhibiting or retarding a disease or condition in an individual who is experiencing or displaying the pathology or symptomatology of the disease or condition; and (3) ameliorating the disease; for example, ameliorating a disease or condition in an individual who is experiencing or displaying the pathology or symptomatology of the disease or condition (i.e., reversing the pathology and/or symptomatology) such as decreasing the severity of disease or completely eliminating/curing the disease. As used herein, treating a disease further includes treating one or more symptoms associated with the disease or condition.

The phrase “disease or condition” also encompasses disorders.

Treatment of the diseases or conditions herein includes treating one or more symptoms associated with diseases and/or conditions associated with Lp-PLA₂ and/or 15-LOX. In some embodiments, the disease or condition associated with Lp-PLA₂ and/or 15-LOX is a cardiovascular disease or condition. In some embodiments, the disease or condition associated with Lp-PLA₂ and/or 15-LOX is an inflammatory disease or condition. In some embodiments, the disease or condition associated with Lp-PLA₂ and/or 15-LOX is a proliferative disease or condition, in particular, cancer. Some examples of diseases or conditions associated with Lp-PLA₂ and/or 15-LOX are provided above and more will be known to those skilled in the art. In one embodiment, the disease or condition associated with Lp-PLA₂ and/or 15-LOX is atherosclerosis.

Advantageously, it is believed that the compounds of the present disclosure may be administered to both non-diabetic and diabetic pations.

As used herein, the term “contacting” refers to the bringing together of indicated moieties in an in vitro system or an in vivo system. For example, “contacting” a an enzyme with a compound of the present disclosure includes the administration of a compound of the present disclosure to an individual or patient, such as a human, having such an enzyme, as well as, for example, introducing a compound of the present disclosure into a sample containing a cellular or purified preparation containing the enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

### Combination Therapies

One or more additional pharmaceutical agents, for example, other pharmaceutical agents for treating a disease or condition associated with Lp-PLA₂- and/or 15-LOX, or other agents, can be used in combination with the compounds of the present disclosure for treatment of Lp-PLA₂- and/or 15-LOX-associated diseases or conditions, such as a cardiovascular, inflammatory or proliferative disease or condition. In some embodiments, a compound of the present disclosure may be used in combination with a cardiovascular agent, such as a statin, acetylcholinesterase (ACE) inhibitor or angiotensin II receptor blocker (ARB). In some embodiments, a compound of the present disclosure may be used in combination with an antiinflammatory agent, such as an NSAID, a COX inhibitor, or a steroid. In some embodiments, a compound of the present disclosure may be used in combination with a cancer chemotherapeutic agent. The one or more additional pharmaceutical agents can be administered to a patient simultaneously or sequentially.

Additive or synergistic effects are desirable outcomes of combining an Lp-PLA₂- and/or 15-LOX inhibitor/antagonist of the present disclosure with one or more additional agent. In some cases, synergy may be achieved by targeting pathways other than Lp-PLA₂ and/or 15-LOX pathways. The additional agents can be combined with the present compounds in a single or continuous dosage form, or the agents can be administered simultaneously or sequentially as separate dosage forms. In some embodiments, one or more additional agents can be administered to a patient in combination with at least one Lp-PLA₂ and/or 15-LOX inhibitor described herein where the additional agents are administered intermittently as opposed to continuously.

### Pharmaceutical Formulations and Dosage Forms

In another aspect, there are provided pharmaceutical compositions a compound as defined herein, or a pharmaceutically acceptable salts thereof, and a pharmaceutically acceptable carrier.

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nano particulate) preparations of the compounds of the invention can be prepared by processes known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and can be generally administered in a pharmaceutically effective amount. For example, the dosage of the active compounds of the invention as employed for the treatment of a patient in need thereof (such as an adult human) may range from 0.1 to 3000 mg per day, depending on the route and frequency of administration. Such a dosage corresponds to 0.001 to 50 mg/kg per day. In some embodiments, the dosage of the active compounds of the invention as employed for the treatment of a patient in need thereof (such as an adult human) may range from 1 to 2000 mg per day, from 1 to 1000 mg per day, from 10 to 1000 mg per day, or from 10 to 500 mg per day. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient can be mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or condition, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents.

### Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

In some embodiments, the present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from ³H, ¹⁴C, ¹²⁵I, ³⁵S and ⁸²Br.

In some embodiments, the labeled compounds of the present invention contain a fluorescent label.

Synthetic methods for incorporating radio-isotopes and fluorescent labels into organic compounds are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind Lp-PLA₂ and/or 15-LOX by monitoring concentration variation when contacting with the enzyme, through tracking the labeling. For another example, a test compound (labeled) can be evaluated for its ability to reduce binding of another compound which is known to bind to Lp-PLA₂ and/or 15-LOX (i.e., standard compound). Accordingly, the ability of a test compound to compete with the standard compound for binding to the Lp-PLA₂ and/or 15-LOX directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

In some embodiments, the present invention also includes pharmaceutical kits useful, for example, in the treatment or prevention of Lp-PLA₂- and/or 15-LOX-associated diseases or conditions, such as atherosclerosis and other cardiovascular diseases. Such kits include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

Some embodiments of the invention will be described in greater detail by way of specific examples. The following examples are offered for illustrative purposes, and are not intended to limit the invention in any manner Those of skill in the art will readily recognize a variety of non-critical parameters which can be changed or modified to yield essentially the same results.

Various compounds are exemplified in the Examples and were found to be inhibitors of Lp-PLA₂ enzyme and/or 15-LOX enzyme according biological assays provided herein. The compounds tested were further shown to exhibit selective inhibition of Lp-PLA₂ enxyme and/or 15-LOX as compared to other enzymes. Some exemplary data for compounds of the present disclosure are shown in the Examples below.

### EXAMPLES

### Example 1

### 1-[1-(4-methylbenzoyl)-2-phenylindolizin-3-yl]ethanone

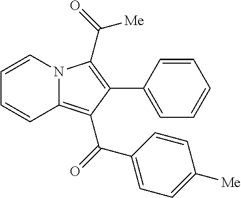 [SMILES: CC(=O)c1c(-c2ccccc2)c(C(=O)c2ccc(C)cc2)c2ccccn12]

The product was obtained as a solid after coupling 4 with 5 as per Scheme 1. ¹H-NMR (300 MHz, CDCl₃) δ 10.00 (d, 1H), δ 7.79 (d, 1H), δ 7.38 (d, 2H), δ 6.94-7.37 (m, 9H), δ 2.27 (s, 3H), δ 1.94 (s, 3H). ESIMS [M+H]=354.2

### Example 2

### 1-[1-(4-isopropylbenzoyl)-2-phenylindolizin-3-yl]ethanone

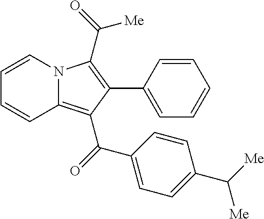 [SMILES: CC(=O)c1c(-c2ccccc2)c(C(=O)c2ccc(C(C)C)cc2)c2ccccn12]

The product was obtained as a solid after coupling 4 with 5 as per Scheme 1. ¹H-NMR (300 MHz, CDCl₃) δ 10.01 (d, 1H), δ 7.93 (d, 1H), δ 6.88-7.39 (m, ¹H), δ 2.75 (m, 1H), δ 1.93 (s, 3H). δ 1.14 (d, 6H). ESIMS [M+H]=382.1

### Example 3

### 1-[1-(4-methoxybenzoyl)-2-phenylindolizin-3-yl]ethanone

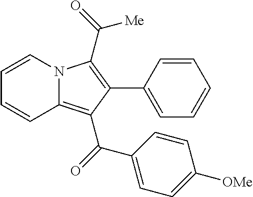 [SMILES: COc1ccc(C(=O)c2c(-c3ccccc3)c(C(C)=O)n3ccccc23)cc1]

The product was obtained as a solid after coupling 4 with 5 as per Scheme 1. ¹H-NMR (300 MHz, CDCl₃) δ 10.00 (d, 1H), δ 7.76 (d, 1H), δ 7.50 (d, 2H), δ 6.97-7.31 (m, 7H), 6.64 (d, 2H), δ 3.76 (s, 3H), δ 1.94 (s, 3H). ESIMS [M+H]=370.2

### Example 4

### 1-[1-(4-(methylthio)benzoyl)-2-phenylindolizin-3-yl]ethanone

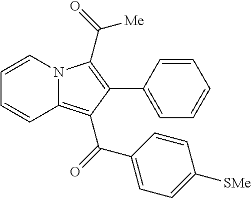 [SMILES: CSc1ccc(C(=O)c2c(-c3ccccc3)c(C(C)=O)n3ccccc23)cc1]

The product was obtained as a solid after coupling 4 with 5 as per Scheme 1. ¹H-NMR (300 MHz, CDCl₃) δ 10.02 (d, 1H), δ 7.82 (d, 1H), δ 7.39 (d, 2H), δ 6.96-7.34 (m, 9H), δ 2.41 (s, 3H), δ 1.94 (s, 3H). ESIMS [M+H]=386.1

### Example 5

### 1-[1-(4-chlorobenzoyl)-2-phenylindolizin-3-yl]ethanone

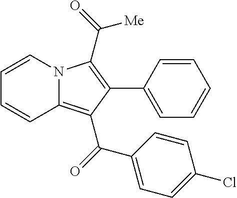 [SMILES: CC(=O)c1c(-c2ccccc2)c(C(=O)c2ccc(Cl)cc2)c2ccccn12]

The product was obtained as a solid after 4 with 5 as per Scheme 1. ¹H-NMR (300 MHz, CDCl₃) δ 10.01 (d, 1H), δ 8.04 (d, 1H) δ 7.03-7.40 (m, 11H), δ 1.93 (s, 3H). ESIMS [M+H]=374.0

### Example 6

### 1-[1-(4-bromobenzoyl)-2-phenylindolizin-3-yl]ethanone

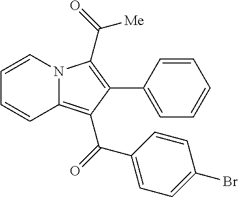 [SMILES: CC(=O)c1c(-c2ccccc2)c(C(=O)c2ccc(Br)cc2)c2ccccn12]

The product was obtained as a solid after coupling 4 with 5 as per Scheme 1. ¹H-NMR (300 MHz, CDCl₃) δ 10.01 (d, 1H), δ 7.92 (dd 1H), δ 7.03-7.45 (m, ¹H), δ 1.93 (s, 3H). ESIMS [M+H]=418.0.

### Example 7

### 1-[1-(4-fluorobenzoyl)-2-phenylindolizin-3-yl]ethanone

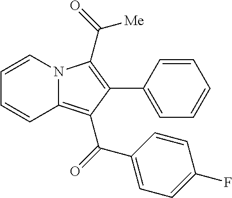 [SMILES: CC(=O)c1c(-c2ccccc2)c(C(=O)c2ccc(F)cc2)c2ccccn12]

The product was obtained as a solid after coupling 4 with 5 as per Scheme 1. ¹H-NMR (300 MHz, CDCl₃) δ 10.01 (d, H), δ 7.98 (d, 1H), δ 7.05-7.45 (m, 9H), δ 6.75 (t, 2H), δ 1.93 (s, 3H). ESIMS [M+H]=358.1

### Example 8

### 1-[2-phenyl-1-(4-(trifluoromethyl)benzoyl)indolizin-3-yl]ethanone

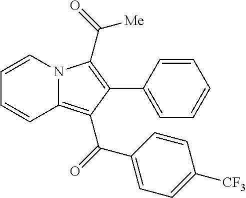 [SMILES: CC(=O)c1c(-c2ccccc2)c(C(=O)c2ccc(C(F)(F)F)cc2)c2ccccn12]

The product was obtained as a solid after coupling 4 with 5 as per Scheme 1. ¹H-NMR (300 MHz, CDCl₃) δ 10.02 (d, 1H), δ 8.24 (d, 1H), δ 6.99-7.37 (m, ¹H), δ 1.89 (s, 3H). ESIMS [M+H]=408.2.

### Example 9

### 1-[1-(3,4-difluorobenzoyl)-2-phenylindolizin-3-yl]ethanone

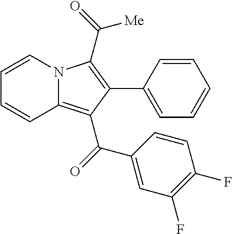 [SMILES: CC(=O)c1c(-c2ccccc2)c(C(=O)c2ccc(F)c(F)c2)c2ccccn12]

The product was obtained as a solid after coupling 4 with 5 as per Scheme 1. ¹H-NMR (300 MHz, CDCl₃) δ 10.01 (d, 1H), δ 8.10 (d, 1H), δ 7.40 (t, 1H), δ 7.06-7.24 (m, 8H), δ 6.81-6.90 (m, 1H) δ 1.93 (s, 3H). ESIMS [M+H]=376.1

### Example 10

### 1-[1-benzoyl-2-propylindolizin-3-yl]ethanone

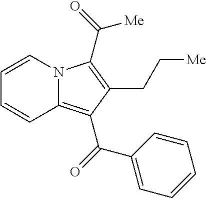 [SMILES: CCCc1c(C(=O)c2ccccc2)c2ccccn2c1C(C)=O]

The product was obtained as a solid after coupling 4 with 5 as per Scheme 1. ¹H-NMR (300 MHz, CDCl₃) δ 9.96 (d, 1H), δ 7.41-7.72 (m, 5H), δ 7.09-7.23 (m, 2H), δ 6.84-6.89 (m, 1H), 3.05-3.10 (m, 2H), 2.76 (s, 3H), 1.61-1.71 (m, 2H), 0.86 (t, 3H). ESIMS [M+H]=306.1

### Example 11

### 1-[1-benzoyl-2-p-tolylindolizin-3-yl]ethanone

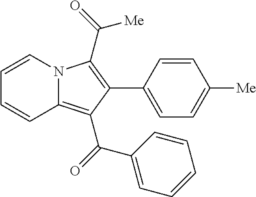 [SMILES: CC(=O)c1c(-c2ccc(C)cc2)c(C(=O)c2ccccc2)c2ccccn12]

The product was obtained as a solid after coupling 4 with 5 as per Scheme 1. ¹H-NMR (300 MHz, CDCl₃) δ 10.01 (d, 1H), δ 7.87 (d, 1H), δ 7.44 (d, 2H), δ 6.95-7.35 (m, 9H), δ 2.24 (s, 3H), δ 1.95 (s, 3H). ESIMS [M+H]=354.2

### Example 12

### 1-[1-benzoyl-2-(4-(trifluoromethyl)phenyl)indolizin-3-yl]ethanone

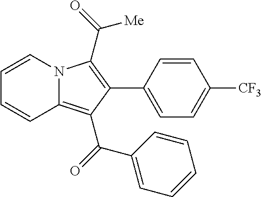 [SMILES: CC(=O)c1c(-c2ccc(C(F)(F)F)cc2)c(C(=O)c2ccccc2)c2ccccn12]

The product was obtained as a solid after coupling 4 with 5 as per Scheme 1. ¹H-NMR (300 MHz, CDCl₃) δ 10.02 (d, H), δ 7.88 (d, 1H), δ 7.07-7.45 (m, ¹H), δ 1.92 (s, 3H). ESIMS [M+H]=408.2

### Example 13

### Inhibition of Lp-PLA₂ and 15-LOX Enxymes by Exemplary Indolizine Derivatives

Lp-PLA₂ Inhibition Assay:

The ability of synthesized indolizine derivatives (6) to inhibit (IC₅₀ values, μM) human Lp-PLA₂ (Cayman Chemical, Ann Arbor, Mich.) was determined based on a UV-spectrometry assay using 96-well plate format. Different concentrations of test compounds (0.001-30 μM) was incubated with Lp-PLA₂ enzyme in 0.1M Tris-HCl (pH 7.2) and in presence of a chromophore precursor [5,5′-dithio-bis-(2-nitrobenzoic acid, DTNB]. The addition of substrate (2-thio-PAF) will cause its hydrolysis by Lp-PLA₂ and the thiol generated was detected by DTNB and measured. The intensity of the color/absorbance developed is inversely proportional to percentage inhibition exhibited by different test compound concentrations. The test compounds stock solution was prepared in assay buffer solution using minimum amount of DMSO (<1%) for solubilization. The concentration of the test compound causing 50% inhibition (IC₅₀, μM) was determined from the concentration-inhibition response curve (duplicate to quadruplicate determinations).

15-LOX Inhibition Assay:

The ability of synthesized indolizine derivatives (6) to inhibit (IC₅₀ values, μM) 15-LOX (Cayman Chemical, Ann Arbor, Mich.) was determined based on a UV-spectrometry assay that detects and measures the hydroperoxides produced in the lipoxygenation reaction using purified lipoxygenase. Stock solution of test compounds was prepared by dissolving in a minimum volume of DMSO (<1%) and was diluted with buffer solution (0.1 M, Tris-HCl pH 7.4). To a 90 μL solution of 15-LOX enzyme in 0.1 M, Tris-HCl pH 7.4 buffer, 10 μL of various concentrations of test drug solutions (0.001-30 μM in a final volume of 210 μL) was added and the lipoxygenase reaction was initiated by the addition of 10 μL (100 μM) of either arachidonic acid (AA) or linoleic acid (LA). After maintaining the 96-well plate on a shaker for 5 min, 100 μL of chromogen was added and retained on a shaker for 5 min. The lipoxygenase activity was determined by measuring absorbance at a wavelength of 490 nm. The color developed is inversely proportional to percentage inhibition due to various test compound concentrations. Percent inhibition was calculated by the comparison of compound-treated to various control incubations. The concentration of the test compound causing 50% inhibition (IC₅₀, μM) was determined from the concentration-inhibition response curve (duplicate to quadruplicate determinations).

The IC₅₀ values for certain example compounds of the invention with respect to Lp-PLA₂ L and 15-LOX are provided in Table 1 as follows.

TABLE 1
Lp-PLA₂ and 15-LOX inhibitory activity of exemplary indolizine
derivatives

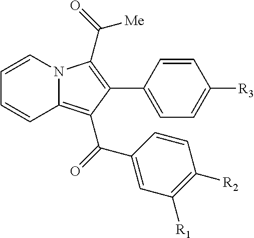

| | | | | | Lp-PLA₂ | |
|---|---|---|---|---|---|---|
| Cmpd | Example | R1 | R2 | R3 | (IC₅₀, nM) | 15-LOX (IC₅₀, μM) |
| 1 | | H | H | H | 510 | >10 |
| 2 | Example 1 | H | Me | H | 30 | 3.58 |
| 3 | Example 2 | H | i-Pr | H | 800 | 4.92 |
| 4 | | H | n-Bu | H | 290 | 6.07 |
| 5 | Example 3 | H | OMe | H | 760 | 5.21 |
| 6 | Example 4 | H | SMe | H | 240 | 3.36 |
| 7 | Example 8 | H | CF₃ | H | 60 | 1.31 |
| 8 | Example 7 | H | F | H | 250 | 1.07 |
| 9 | Example 6 | H | Br | H | 940 | 1.25 |
| 10 | Example 5 | H | Cl | H | 700 | 1.27 |
| 11 | Example 11 | H | H | Me | 20 | 3.45 |
| 12 | Example 9 | F | F | H | 790 | 5.21 |

when the experiment limit is set as “a” and the IC₅₀ measurement of the example compound exceeds the limit, then the IC₅₀ data is shown as “>a”

### Example 14

### Selectivity of Exemplary Indolizine Derivatives for Lp-PLA2 and 15-LOX

Inhibition assays were carried out to determine the selectivity of indolizine derivatives for Lp-PLA2 and 15-LOX relative to sPLA₂, COX-1 and COX-2.

Phospholipase A₂ (PLA2) Inhibition Assay:

Phospholipase inhibition by synthesized compounds toward human Lp-PLA2, sPLA2 IIA and IV (Cayman Chemical Company, Ann Arbor, Mich.) are evaluated using a 96-well plate format. In this assay, different concentrations of test compounds (0.001-30 μM) are incubated with the PLA2 enzymes. The substrate hydrolysis by PLA2 is monitored by UV-spectroscopy. The intensity of the color developed is inversely proportional to percentage inhibition exhibited by different test compound concentrations. The concentration of the test compound causing 50% inhibition (IC₅₀, μM) is determined from the concentration-inhibition response curve. Isothermal calorimetry (ITC) experiments using will be carried out to determine binding constant (KB) and dissociation constant (K_{D}) of Lp-PLA2 inhibitors (Ann. Rev. Biophy. 37, 135-151, 2008; J. Am. Chem. Soc. 125, 10570-10579, 2003)]. Reference compounds (eg: varespladib and darapladib) will be used for comparison.

Lipoxygenase (LOX) Inhibition Assays:

The ability of the test compounds to inhibit (IC₅₀ values, μM) 5- and 15-lipoxygenase (human; Cayman Chemical, Ann Arbor) are determined by a UV-spectrometry based assay that measures the hydroperoxides produced in the lipoxygenation reaction. To a solution of 5, 12 or 15-LOX enzymes, various concentrations of test drug solutions (0.001-30 μM) are added and the lipoxygenase reaction is initiated by the addition of substrate (arachidonic acid or linoleic acid) in a 96-well plate. The concentration of the test compound causing 50% inhibition (IC₅₀, μM) is determined from the concentration-inhibition response curve. Reference compounds will be used for comparison (eg: nordihydroguaiaretic acid, zileuton).

Cyclooxygenase (COX) Inhibition Assays:

The ability of the test compounds to inhibit (IC₅₀ values, μM) human COX-1 and COX-2 (Cayman Chemical, Ann Arbor) is determined PGF_{2α}, produced from COX reaction is measured by enzyme immunoassay. Either COX-1 or COX-2 enzyme in the presence of heme and various concentrations of test compound solutions (0.01-10 μM) is incubated after which substrate arachidonic acid is added. The concentration of the test compound causing 50% inhibition (IC₅₀, μM) is calculated from the concentration-inhibition response curve.

The IC₅₀ values for certain exemplary indolizine compounds with respect to Lp-PLA₂, 15-LOX, sPLA₂, COX-1 and COX-2 are provided in Table 2 as follows, with reference to Scheme 4 below.

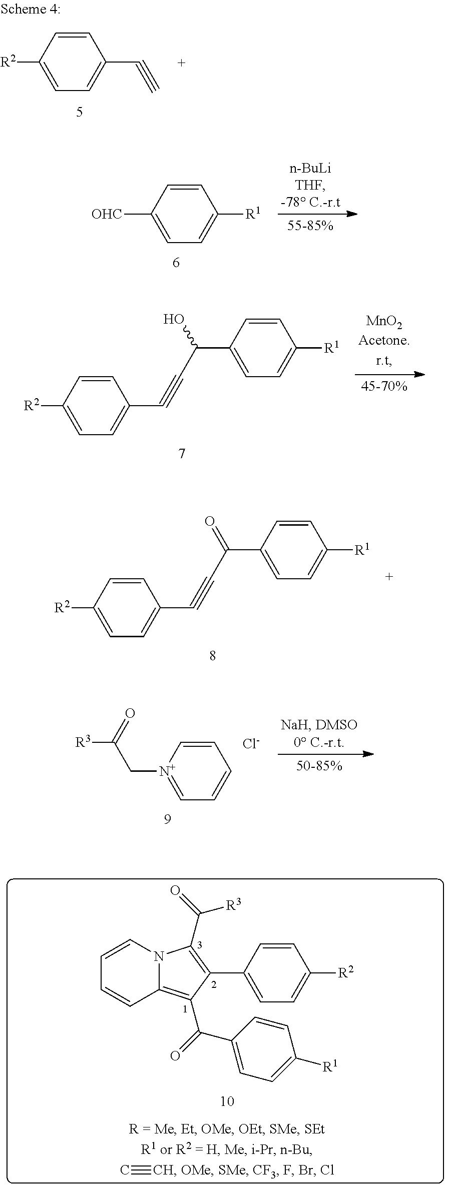

TABLE 2
Selectivity of select indolizine derivatives (10a-j) for Lp-PLA₂ and 15-LOX

| | | | | | | Selectivity | | | |
|---|---|---|---|---|---|---|---|---|---|
| | | | | Lp-PLA₂ | sPLA₂ IIA | Index | 15-LOX | COX-1 | COX-2 |
| | | | | Inhibition | Inhibition | (sPLA₂/Lp- | Inhibition | Inhibition | Inhibition |
| Compd | R1 | R2 | R3 | (IC₅₀, μM) | (IC₅₀, μM | PLA₂) | (IC₅₀, μM) | (IC₅₀, μM | (IC₅₀, μM |
| 10a | H | H | Me | 0.51 | >10 | >19.6 | >10 | >10 | >10 |
| 10b | Me | H | Me | 0.02 | >10 | >500 | 3.58 | >10 | >10 |
| 10c | i-Pr | H | Me | 0.80 | >10 | >12.5 | 4.92 | >10 | >10 |
| 10d | n-Bu | H | Me | 0.30 | >10 | >33.3 | 6.07 | >10 | >10 |
| 10e | OMe | H | Me | 0.76 | >10 | >13.1 | 5.21 | >10 | >10 |
| 10f | SMe | H | Me | 0.25 | >10 | >40 | 3.36 | >10 | >10 |
| 10g | CF₃ | H | Me | 0.06 | >10 | >166.6 | 1.31 | >10 | >10 |
| 10h | F | H | Me | 0.57 | >10 | >17.5 | 1.07 | >10 | >10 |
| 10i | Br | H | Me | 0.70 | >10 | >14.2 | 1.25 | >10 | >10 |
| 10j | Cl | H | Me | 0.94 | >10 | >10.6 | 1.27 | >10 | >10 |

Various modifications of the invention, in addition to those described herein, will be apparent to those skilled in the art from the foregoing description. Such modifications are also intended to fall within the scope of the appended claims. Each reference cited in the present application is herein incorporated by reference in its entirety.

1. A method of treating a disease or condition associated with at least one of a lipoprotein associated phospholipase A₂ (Lp-PLA₂) and a 15-lipoxygenase (15-LOX) in a patient comprising administering to said patient a therapeutically effective amount of a compound of Formula I: 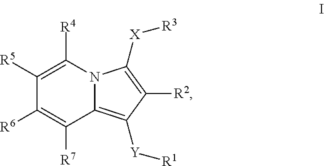
or a pharmaceutically acceptable salt thereof, wherein:
X and Y are independently C(O), C(S), NH, NR^{a}, S, or O, where Y can be present or absent;
R¹ is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO₂, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
with the proviso that when Y is C(O), R¹ is not C(O)NH₂, C(O)NHR^{d} or C(O)NR^{c}R^{d};
R² is a non-interfering substituent selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NO₂, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
R³ is a non-interfering substituent selected from C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}, OC(O)NH₂, OC(O)NHR^{d}, OC(O)NR^{d}R^{d}, OC(O)OR^{d}, C(O)R^{e}, C(O)NH₂, C(O)NHR^{d}, C(O)NR^{d}R^{d}, C(O)OR^{d}, NH₂, NR^{f}H, NR^{f}R^{f}, NR^{e}C(O)NH₂, NR^{e}C(O)R^{d}, NR^{e}C(O)OR^{d} and NR^{e}C(O)NR^{e}R^{e},
wherein each C₁₋₆ alkyl, C₁₋₆ haloalkyl, C₂₋₆ alkenyl, C₂₋₆ alkynyl, C₃-C₄ cycloalkyl, OR^{d}, SR^{d}, OC(O)R^{e}, OC(O)NHR^{d}, OC(O)NR^{d}R^{d}, OC(O)OR^{d}, C(O)R^{e}, C(O)NHR^{d}, C(O)NR^{d}R^{d}, C(O)OR^{d}, NR^{f}H, NR^{f}R^{f}, NR^{e}C(O)NH₂, NR^{e}C(O)R^{d}, NR^{e}C(O)OR^{d} and NR^{e}C(O)NR^{e}R^{e} is optionally substituted by one or more substituents independently selected from halogen, C₁₋₆ alkyl, C₁₋₆ haloalkyl and C₃-C₄ cycloalkyl;
with the proviso that when X is C(O), R³ is not C(O)NH₂, C(O)NHR^{d} or C(O)NR^{d}R^{d};
R⁴, R⁵, R⁶ and R⁷ are each non-interfering substituents independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NfIR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{e}R^{e}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b};
R^{a} is C₁₋₆ alkyl, C₁₋₆ haloalkyl, C₂₋₆ alkenyl, C₂₋₆ alkynyl, C₃₋₄ cycloalkyl, alkylchalcogen, arylchalcogen, aryl, cycloalkyl, heteroaryl, heterocycloalkyl, arylalkyl, or heteroarylalkyl;
R^{b} is H, C₁₋₆ alkyl, C₁₋₆ haloalkyl, C₂₋₆ alkenyl, C₂₋₆ alkynyl, C₃₋₄ cycloalkyl, cyclopropyl, amino, alkylchalcogen, arylchalcogen, aryl, cycloalkyl, heteroaryl, heterocycloalkyl, arylalkyl, or heteroarylalkyl;
R^{c} is C₁₋₆ alkyl, C₁₋₆ haloalkyl, C₂₋₆ alkenyl, C₂₋₆ alkynyl, C₃₋₄ cycloalkyl, alkylchalcogen, arylchalcogen, aryl, cycloalkyl, heteroaryl, heterocycloalkyl, arylalkyl, or heteroarylalkyl;
R^{d} is C₁₋₆ alkyl, C₁₋₆ haloalkyl, C₂₋₆ alkenyl, C₂₋₆ alkynyl, C₃₋₄ cycloalkyl, or amino;
R^{e} is H, C₁₋₆ alkyl, C₁₋₆ haloalkyl, C₂₋₆ alkenyl, C₂₋₆ alkynyl, C₃₋₄ cycloalkyl, cyclopropyl, or amino; and
R^{f} is C₁₋₆ alkyl, C₁₋₆ haloalkyl, C₂₋₆ alkenyl, C₂₋₆ alkynyl, or C₃₋₄ cycloalkyl.

2. The method of claim 1, wherein
X and Y are independently C(O) or C(S);
R¹ is selected from C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a}, is optionally substituted by one or more substituents independently selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};
R² is selected from C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a}, is optionally substituted by one or more substituents independently selected from halogen, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a} and SR^{a};
R³ is selected from C₁₋₆ alkyl, C₁₋₆ haloalkyl, C₂₋₆ alkenyl, C₂₋₆ alkynyl, C₃-C₄ cycloalkyl, OR^{d} and SR^{d}; and
R⁴, R⁵, R⁶, R⁷, R^{a}, R^{b}, R^{c} R^{d}, R^{e} and R^{f} are as defined in claim 1.

3. The method of claim 1, wherein
X and Y are independently C(O) or C(S);
R¹ is selected from C₁₋₆ alkyl, C₁₋₆ haloalkyl, C₆-C₁₀ aryl, C₃-C₆ cycloalkyl, and C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, or C₅ heteroaryl;
R² is selected from C₁₋₆ alkyl, C₁₋₆ haloalkyl, C₆-C₁₀ aryl, C₃-C₆ cycloalkyl, and C₅-C₁₂ heteroaryl, wherein each is optionally substituted by one or more substituents independently selected from halogen, C₁₋₄ alkyl, C₁₋₄ haloalkyl, C₆ aryl, C₃-C₄ cycloalkyl, C₅ heteroaryl, OR^{a} and SR^{a}; where R^{a} is C₁₋₄ alkyl, C₁₋₄ haloalkyl, C₆ aryl, C₃-C₄ cycloalkyl, or C₅ heteroaryl;
R³ is selected from C₁₋₄ alkyl, C₁₋₄ haloalkyl, C₃-C₄ cycloalkyl, OR^{d} and SR^{d}; wherein R^{d} is C₁₋₄ alkyl, C₁₋₄ haloalkyl, or C₃-C₄ cycloalkyl;
R⁴, R⁵, R⁶ and R⁷ are independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, alkylchalcogen, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b},
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b};
R^{a}, R^{b}, R^{c}, R^{d}, R^{e} and R^{f} are as defined in claim 1.

4. (canceled)

5. (canceled)

6. (canceled)

7. The method of claim 1 wherein the compound is a compound of Formula II: 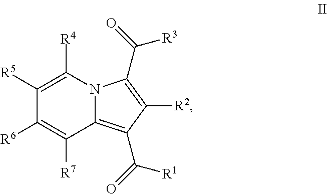
or a pharmaceutically acceptable salt thereof, wherein R¹-R⁷ are as defined in claim 1.

8. (canceled)

9. (canceled)

10. (canceled)

11. The method of claim 1 wherein the compound is a compound of Formula VI: 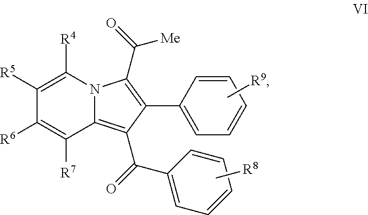
or a pharmaceutically acceptable salt thereof, wherein R⁴-R⁷ are as defined in claim 1 and wherein R⁸ and R⁹ each represent 1, 2 or 3 non-interfering ring substituents independently selected from halogen, OH, NO₂, optionally substituted C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}

12. (canceled)

13. (canceled)

14. (canceled)

15. (canceled)

16. The method of claim 1 wherein the compound is a compound of Formula VII: 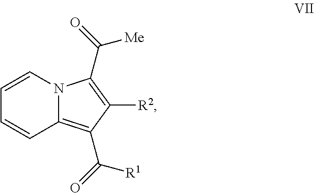
or a pharmaceutically acceptable salt thereof, wherein R¹ and R² are as defined in claim 1.

17. (canceled)

18. The method of claim 16, wherein R¹ is C₆ aryl substituted with 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

19. (canceled)

20. The method of claim 16, wherein R² is C₆ aryl substituted with 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

21. (canceled)

22. (canceled)

23. (canceled)

24. (canceled)

25. (canceled)

26. (canceled)

27. (canceled)

28. (canceled)

29. (canceled)

30. The method according to claim 1, wherein:
X and Y are independently C(O) or C(S);
R¹ and R² are independently selected from C₁₋₆ alkyl, C₆-C₁₀ aryl, and C₅-C₁₂ heteroaryl, wherein each is optionally substituted by one or more substituents independently selected from halogen, C₁₋₄ alkyl, C₁₋₄ haloalkyl, OR^{a} and SR^{a}; where R^{a} is C₁₋₄ alkyl, and C₁₋₄ haloalkyl; and
R³ is C₁₋₄ alkyl; and
R⁴-R⁷ are each H.

31. The method according to claim 30, wherein:
X and Y are C(O);
R¹ and R² are independently selected from C₁₋₆ alkyl and C₆ aryl, wherein each is optionally substituted by one or more substituents independently selected from halogen, C₁₋₄ alkyl, C₁₋₄ haloalkyl, OMe, OEt, SMe and SEt; and
R³ is methyl; and
R⁴-R⁷ are each H.

32. (canceled)

33. The method of claim 1, wherein the compound has the structure of Formula VI: 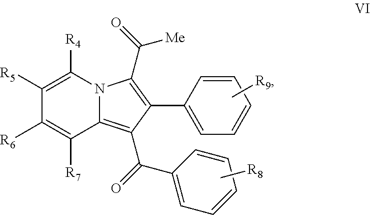
or a pharmaceutically acceptable salt thereof, wherein
R⁴, R⁵, R⁶ and R⁷ are independently selected from H, OH, halogen, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, alkylchalcogen, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b};
wherein each of C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NHR^{a}, OC(O)NR^{a}R^{a}, OC(O)OR^{a}, C(O)R^{b}, C(O)NHR^{a}, C(O)NR^{a}R^{a}, C(O)OR^{a}, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}, is optionally substituted by one or more substituents independently selected from halogen, OH, CN, NO₂, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, OR^{a}, SR^{a}, OC(O)R^{b}, OC(O)NH₂, OC(O)NHR^{a}, OC(O)NR^{a}R^{a}, OC(O)OR^{a}, C(O)R^{b}, C(O)NH₂, C(O)NHR^{a}, C(O)NR^{a}R^{a}, C(O)OR^{a}, NH₂, NR^{c}H, NR^{c}R^{c}, NR^{b}C(O)NH₂, NR^{b}C(O)R^{a}, NR^{b}C(O)OR^{a} and NR^{b}C(O)NR^{b}R^{b}; and
R⁸ and R⁹ each represent 1, 2 or 3 non-interfering ring substituents independently selected from H, halogen, OH, C₁₋₆ alkyl, C₁₋₆ haloalkyl, C₆-C₁₀ aryl, C₃-C₆ cycloalkyl, and C₅-C₁₂ heteroaryl, wherein each of C₁₋₆ alkyl, C₁₋₆ haloalkyl, C₆-C₁₀ aryl, C₃-C₆ cycloalkyl, and C₅-C₁₂ heteroaryl is optionally substituted by one or more substituents independently selected from halogen, OH, C₁₋₄ alkyl, C₁₋₄ haloalkyl, C₆ aryl, C₃-C₄ cycloalkyl, C₅ heteroaryl, OR^{a} and SR^{a};
where R^{a} is C₁₋₄ alkyl, C₁₋₄ haloalkyl, C₆ aryl, C₃-C₄ cycloalkyl, or C₅ heteroaryl.

34. The method of claim 33, wherein R⁸ and R⁹ each represent 1 or 2 ring substituents selected from H, halogen, —O(C₁-C₄ alkyl), —S(C₁-C₄ alkyl), methyl, ethyl, n-propyl, isopropyl, n-butyl, t-butyl, and CF³.

35. (canceled)

36. (canceled)

37. (canceled)

38. (canceled)

39. (canceled)

40. (canceled)

41. The method of claim 1, wherein the compound is selected from the group consisting of:
1-[1 (4-methylbenzoyl)-2-phenylindolizin-yl]ethanone;
1-[1 (4-isopropylbenzoyl)-2-phenylindolizin-yl]ethanone;
1-[1 (4-methoxybenzoyl)-2-phenylindolizin-yl]ethanone;
1-[1 (4-(methylthio)benzoyl)-2-phenylindolizin-3-yl]ethanone;
1-[1 (4-chlorobenzoyl)-2-phenylindolizin-3-yl]ethanone;
1-[1 (4-bromobenzoyl)-2-phenylindolizin-3 yl]ethanone;
1-[1 (4-fluorobenzoyl)-2-phenylindolizin-yl]ethanone;
1-[2-phenyl-1-(4-(trifluoromethyl)benzoyl)indolizin-3-yl]ethanone;
1-[1-(3,4-difluorobenzoyl)-2-phenylindolizin-3-yl]ethanone;
1-[1-benzoyl-2-propylindolizin-3-yl]ethanone;
1-[1-benzoyl-2-p-tolylindolizin-3-yl]ethanone; and
1-[1-benzoyl-2-(4-(trifluoromethyl)phenyl)indolizin-3-yl]ethanone.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method of claim 1, wherein the disease or condition is associated with an Lp-PLA₂ and a 15-LOX.

60. The method of claim 1 wherein the disease or condition is a cardiovascular disease or condition, an inflammatory disease or condition or cancer.

61. The method of claim 60 wherein the cardiovascular disease or condition is atherosclerosis, stroke, myocardial infarction, acute coronary syndrome, coronary heart disease, peripheral arterial disease or reperfusion injury.

62. (canceled)

63. The method of claim 60 wherein the inflammatory disease or condition is chronic/acute inflammation, rheumatoid arthritis, psoriasis or asthma.

64. The method of claim 60 wherein the cancer is prostate, pancreatic or colorectal cancer.

65. The method of claim 1 wherein the disease or condition is diabetes.

66. (canceled)

67. (canceled)

68. A method of preparing a compound of Formula I, comprising:
reacting, in a solution of NaH in DMSO, a compound of formula (i) 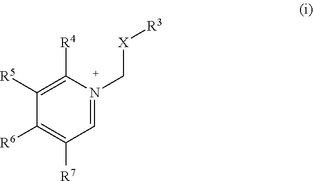
in the presence of a suitable counterion, with a compound of formula (ii) 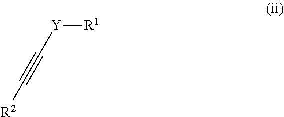
to form a compound of Formula I 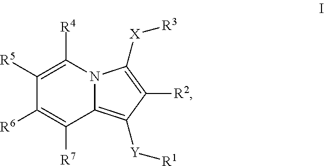
wherein X and Y are independently C(O) or C(S), and wherein each of R¹ to R⁷ are as defined in claim 1.

69. (canceled)

70. (canceled)

71. (canceled)

72. (canceled)

73. (canceled)

74. (canceled)

75. (canceled)

76. (canceled)

77. (canceled)

78. (canceled)

79. (canceled)
